(19) United States
(12) Patent Application Publication
(10) Pub. No.: US 2007/0082339 A1
(43) Pub. Date: Apr. 12, 2007

(54) Methods and compositions for screening for HDAC inhibitory activity

(76) Inventors: Dalia Cohen, Winchester, MA (US); Shaowen Wang, Andover, MA (US)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 11/246,802

(22) Filed: Oct. 7, 2005

(51) Int. Cl. C12Q 1/68; G01N 33/567

(52) U.S. Cl. 435/6; 435/7.2

(57) ABSTRACT

The invention relates to the use of gene and protein expression levels as a marker for HDAC inhibition. Also disclosed are in vivo and in vitro methods for screening a compound for HDAC inhibitory activity, as well as methods for monitoring the therapeutic efficacy of an HDAC inhibitor in a subject in vivo and for determining resistance to an HDAC inhibitor in vitro or in vivo.

### BACKGROUND OF THE INVENTION

The interplay between acetylation and deacteylation of histones has an effect on the active state of genes. Histone acetyltransferases (HATs) are enzymes which are important for transcription or gene activation. Histone deacetylases (HDACs) are enzymes that are important regulators of chromatin structure and transcription known to modulate cell cycle, hormone signalling and development. The regulation or deregulation of these enzymes has been linked to progression of cancers such as leukaemia, colorectal and breast cancers as well as other diverse human disorders. For example, inhibitors of histone deacetylase such as phenylbutyrate and trichostatin A have shown promise in the treatment of promyelocytic leukemia. In addition, butyrate decreases the expression of pro-inflammatory cytokines TNF-α, TNF-β, IL-6, and IL1 -β most likely through inhibition of NFkB activation and inhibition of histone deacetylases. Also, trapoxin A (Tpx A), a microbially derived cyclotetrapeptide (Itazaki et al. J. Antibiot. 43(12):1524-1532 (1990)) has been shown to bind to and potently inhibit histone deacetylase 1 (HDACl) (Tauton et al. Science 272:408-411 (1996)).

Upon acetylation or deacetylation of histones, a cascade of chemical and structural reactions occur inducing or inhibiting various signal and expression pathways and formation of protein and regulatory complexes. The challenge is to attribute specific HDAC complexes to cellular function, and to identify molecules able to block the activity of functions such as cell proliferation in cancer. Meeting this challenge is likely to produce therapies with improved efficacy and selectivity, and an ability to target the expression of selected genes. There remains a need for identifying and characterizing the effects of HDAC transcriptional activity and its relation to specific diseases.

### SUMMARY OF THE INVENTION

The invention provides methods for screening for HDAC inhibition and HDAC inhibitors in vitro and in vivo by detecting expression levels of any one or more genes disclosed in Table 1. The invention also relates to a method for monitoring the therapeutic efficacy of an HDAC inhibitor in a subject in vivo as well as in vivo and in vitro methods to determine resistance to an HDAC inhibitor.

In one aspect, the invention pertains to a method for screening for HDAC activity in cells in vitro comprising a) detecting expression levels of any one or more genes selected from the group consisting of those disclosed in Table 1 in said cells and in control cells and b) comparing expression levels of genes in cells with expression levels corresponding to genes from control cells wherein a difference in expression levels between the cells and control cells indicates said cells contain HDAC activity activity. In one aspect, the expression levels detected are mRNA, cDNA or proteins encoded by such genes. In yet another aspect, the HDAC activity detected is produced as a result of HDAC inhibition.

In another aspect, the invention pertains to a method for screening for HDAC inhibition in a subject in vivo comprising a). detecting expression levels of any one ore more genes selected from the group consisting of those disclosed in Table 1 in said subject in vivo and in a control subject known not to possess conditions related to abnormal HDAC activity; and b). comparing the expression levels from the subject with expression levels from the control subject wherein a difference in expression levels between the subject relative to expression levels in the control subject indicates that HDAC activity is inhibited in said subject in vivo. In one aspect, the expression levels detected are mRNA, cDNA or proteins encoded by such genes. In yet another aspect, the HDAC activity detected is produced as a result of HDAC inhibition.

In another aspect the invention pertains to a method for screening a compound suspected of possessing HDAC inhibitory activity in vitro, comprising a). administering a test compound to cells in vitro to obtain treated cells; b). assaying for expression of any one or more genes selected from the group consisting of those disclosed in Table 1 in the treated cells and in control cells to which no compound has been administered; and c) comparing expression levels of said genes in the treated cells and in the control cells wherein a difference in expression levels between the treated cells and the control cells indicates whether a compound possesses HDAC inhibitory activity. In one aspect, the expression levels detected are mRNA, cDNA or proteins encoded by such genes. In yet another aspect, the HDAC activity detected is produced as a result of HDAC inhibition.

In another aspect the invention pertains to a method for screening a compound for HDAC inhibitory activity in a subject in vivo, comprising a). assaying for expression levels of any one or more genes selected from the group consisting of those disclosed in Table I in said subject; b). administering a compound to said subject; c). reassaying for expression levels of any one or more genes selected from the group consisting of those disclosed in Table 1 in said subject after administration of the compound; and d) comparing said expression levels in said subject before and after administration of the compound wherein a difference in expression levels in said subject after administration of the compound compared to levels before compound administration indicates whether said compound possesses HDAC inhibitory activity

In yet another aspect, the invention pertains to a method for monitoring the therapeutic efficacy of a known HDAC inhibitor in a subject comprising a) detecting expression levels of any one or more genes selected from the group consisting of those disclosed in Table 1 and proteins encoded thereby in said subject before and after treatment with said HDAC inhibitor; and b). comparing said expression levels in said subject wherein a difference in expression levels in said subject after treatment compared to expression levels before treatment indicate that the HDAC inhibitor is therapeutically effective.

In still another aspect, the invention relates to a method to determine sensitivity of a cell to a known HDAC inhibitor comprising a) administering said HDAC inhibitor to cells in vitro; b) screening for expression levels of any one or more genes selected from the group consisting of those disclosed in Table 1 in said cells and in control cells to which no HDAC inhibitor has been administered, and c) comparing levels expression levels of any one or more genes selected from the group consisting of those disclosed in Table 1 in said cells and in said control cells wherein a difference in expression level of any one or more genes disclosed in Table 1 indicates the sensitivity of the said cells to the HDAC inhibitor. In another aspect, the absence in expression of one or more of the genes disclosed in Table 1 indicates the resistance of the said cells to the HDAC inhibitor.

In another aspect, the invention relates to a method to determine resistance of a subject to a known HDAC inhibitor comprising a) detecting expression levels of any one or more genes selected from the group consisting of those disclosed in Table 1 in said subject before and after treatment with said HDAC inhibitor wherein resistance to HDAC inhibition is associated with a lack of expression of any one or more genes selected from those disclosed in Table 1.

In a related aspect the invention relates to a method of diagnosing a condition related to nonphysiological cellular proliferation susceptible to treatment with HDAC inhibitory agents, which comprises measuring in cells of the subject which exhibits the condition a modulation in expression levels of any one or more genes selected from the group consisting of those disclosed in Table 1 The method preferably takes place ex vivo. In one aspect the expression levels of genes disclosed in Table 2 are reversed for conditions wherein a patient does not respond to HDAC inhibition or is resistant to such treatment. Thus, the genes of the invention may also be used as biomarkers of HDAC inhibitor compounds.

In another aspect the invention relates to a method of contributing to the diagnosis or prognosis of, or to developing a therapeutic strategy for a subject having a condition associated with non-physiological cellular proliferation condition susceptible to treatment with HDAC inhibitory agents comprising comparing expression levels of any one or more genes selected from the group disclosed in Table 1 in a sample obtained from said subject relative to expression levels of a control subject known not to have said condition.

In another related aspect of any one or more genes selected from the group consisting of those disclosed in Table 1 is used as a biomarker for HDAC inhibitory activity. The invention is related to a method for treating a condition in a subject wherein the condition is one for which administration of HDAC inhibitors is indicated, comprising a) administering a compound to the subject, b) obtaining the gene expression profile from the subject wherein the gene expression profile comprises the gene expression pattern of one or more genes selected from the group consisting of those disclosed in Table 1 and proteins encoded therefrom, whereby the expression patterns of the genes or proteins are a consequence of administration of the compound, and c) comparing the gene expression profile of the subject to whom the compound was administered to a biomarker gene expression profile indicative of efficacy of treatment by an HDAC inhibitor, wherein a difference in the gene expression profile of the subject to whom the compound was administered to the biomarker gene expression profile is indicative of efficacy of treatment with the compound. In a further aspect, the biomarker gene expression profile is the baseline gene expression profile of the subject before administration of the compound.

In related embodiments of the methods discussed above, protein levels and/or mRNA levels of any one or more genes selected from the group consisting of those disclosed in Table 1, may be assayed.

### DETAILED DESCRIPTION OF THE INVENTION

It is contemplated that the invention described herein is not limited to the particular methodology, protocols, and reagents described as these may vary. It is also to be understood that the terminology used herein is for the purpose of describing particular embodiments only, and is not intended to limit the scope of the present invention in any way.

Unless defined otherwise, all technical and scientific terms used herein have the same meanings as commonly understood by one of ordinary skill in the art to which this invention belongs. Although any methods and materials similar or equivalent to those described herein can be used in the practice or testing of the present invention, the preferred methods and materials are now described. All publications mentioned herein are incorporated by reference for the purpose of describing and disclosing the materials and methodologies that are reported in the publication which might be used in connection with the invention.

In practicing the present invention, many conventional techniques in molecular biology are used. These techniques are well known and are explained in, for example, Current Protocols in Molecular Biology, Volumes I, II, and III, 1997 (F. M. Ausubel ed.); Sambrook et al., 1989, Molecular Cloning: A Laboratory Manual, Second Edition, Cold Spring Harbor Laboratory Press, Cold Spring Harbor, N.Y.; DNA Cloning: A Practical Approach, Volumes I and II, 1985 (D. N. Glover ed.); Oligonucleotide Synthesis, 1984 (M. L. Gait ed.); Nucleic Acid Hybridization, 1985, (Hames and Higgins); Transcription and Translation, 1984 (Hames and Higgins eds.); Animal Cell Culture, 1986 (R. I. Freshney ed.); Immobilized Cells and Enzymes, 1986 (IRL Press); Perbal, 1984, A Practical Guide to Molecular Cloning; the series, Methods in Enzymology (Academic Press, Inc.); Gene Transfer Vectors for Mammalian Cells, 1987 (J. H. Miller and M. P. Calos eds., Cold Spring Harbor Laboratory); and Methods in Enzymology Vol. 154 and Vol. 155 (Wu and Grossman, and Wu, eds., respectively).

As used herein and in the appended claims, the singular forms “a”, “an”, and “the” include plural reference unless the context clearly dictates otherwise. Thus, for example, reference to the “antibody” is a reference to one or more antibodies and equivalents thereof known to those skilled in the art.

“Modulation” by a compound or modulation of expression levels of genes or proteins associated with HDAC activity includes, but is not limited to, the ability of a substance to alter, by inhibiting or enhancing the activity and/or inhibiting or enhancing the expression of any one or more of the genes selected from the group consisting of those disclosed in Table 1 and proteins encoded therefrom. The invention also contemplates identification of pathways and expression levels modulated indirectly as a result of inhibition of HDAC activity including genes expressed upstream or downstream from of genes disclosed in Table 1. Thus, in one aspect, the

“Nucleic acid sequence” or “gene” as used herein, refer to an oligonucleotide, nucleotide or polynucleotide, and fragments or portions thereof, and to DNA or RNTA of genomic or synthetic origin that may be single or double stranded, and represent the sense or antisense strand.

The term “antisense” as used herein, refers to nucleotide sequences which are complementary to a specific DNA or RNA sequence. The term “antisense strand” is used in reference to a nucleic acid strand that is complementary to the “sense” strand. Antisense molecules may be produced by any method, including synthesis by ligating the gene(s) of interest in a reverse orientation to a viral promoter which permits the synthesis of a complementary strand. Once introduced into a cell, this transcribed strand combines natural sequences produced by the cell to form duplexes. These duplexes then block either the further transcription or translation. The designation “negative ” is sometimes used in reference to the antisense strand, and “positive” is sometimes used in reference to the sense strand.

As contemplated herein, antisense oligonucleotides, triple helix DNA, RNA aptamers, siRNA, ribozymes and double or single stranded RNA are designed to inhibit gene or protein expression such that the chosen nucleotide sequence of the protein to which the inhibitory molecule is designed can cause specific inhibition of endogenous protein production. Additionally, ribozymes can be synthesized to recognize specific nucleotide sequences of a protein of interest and cleave it (Cech. J. Amer. Med Assn. 260:3030 (1988)). Techniques for the design of such molecules for use in targeted inhibition of gene expression are well known to one of skill in the art.

The term “sample” or “biological sample” as used herein, is used in its broadest sense. A biological sample from a subject may comprise blood, urine or other biological material with which activity or gene expression of proteins may be assayed.

As used herein, the term “antibody” refers to intact molecules as well as fragments thereof, such as Fa, F(ab′)₂ and Fv, which are capable of binding the epitopic determinant. Antibodies that bind polypeptides disclosed herein can be prepared using intact polypeptides or fragments containing small peptides of interest as the immunizing antigen. The polypeptides or peptides used to immunize an animal can be derived from the translation of RNA or synthesized chemically, and can be conjugated to a carrier protein, if desired. Commonly used carriers that are chemically coupled to peptides include bovine serum albumin and thyroglobulin. The coupled peptide is then used to immunize an animal (e.g., a mouse, a rat or a rabbit).

The term “humanized antibody” as used herein, refers to antibody molecules in which amino acids have been replaced in the non-antigen binding regions in order to more closely resemble a human antibody, while still retaining the original binding ability.

The individual proteins/polypeptides referred to herein include any and all forms of these proteins including, but not limited to, partial forms, isoforms, variants, precursor forms, the full length protein, fusion proteins containing the sequence or fragments of any of the above, from human or any other species. Protein homologs or orthologs which would be apparent to one of skill in the art are included in this definition. It is also contemplated that the term refers to proteins isolated from naturally occurring sources of any species such as genomic DNA libraries as well as genetically engineered host cells comprising expression systems, or produced by chemical synthesis using, for instance, automated peptide synthesizers or a combination of such methods. Means for isolating and preparing such polypeptides are well understood in the art.

A peptide mimetic is a synthetically derived peptide or non-peptide agent created based on a knowledge of the critical residues of a subject polypeptide which can mimic normal polypeptide function. Peptide mimetics can disrupt binding of a polypeptide to its receptor or to other proteins and thus interfere with the normal function of a polypeptide

A “therapeutically effective amount” is the amount of drug sufficient to treat, prevent or ameliorate pathological conditions related to abnormal HDAC activity.

“Subject” refers to any human or nonhuman organism.

A “host cell,” as used herein, refers to a prokaryotic or eukaryotic cell that contains heterologous DNA that has been introduced into the cell by any means, e.g., electroporation, calcium phosphate precipitation, microinjection, transformation, viral infection, and the like.

The term “isolated” means that the material is removed from its original environment (e.g., the natural environment if it is naturally occurring). For example, a naturally-occurring polynucleotide or polypeptide present in a living animal is not isolated, but the same polynucleotide or polypeptide, separated from some or all of the coexisting materials in the natural system, is isolated, even if subsequently reintroduced into the natural system. Such polynucleotides could be part of a vector and/or such polynucleotides or polypeptides could be part of a composition, and still be isolated in that such vector or composition is not part of its natural environment.

It has been found that the expression level of genes selected from the group consisting of those disclosed in Table 1 and proteins encoded therefrom are modulated in mammalian cells exposed to HDAC inhibitors. Because the expression levels of these genes and/or proteins is consistently down regulated or upregulated by a significant fold increase or decrease, depending the specific group of genes, the data obtained herein indicate that the characteristic expression levels of any one or more genes or proteins disclosed herein can be used as markers to determine HDAC inhibition or activity in a biological system.

Data disclosed herein indicate that HDAC inhibition is associated with altered expression levels of any one of the genes disclosed in Table 1 and proteins encoded therefrom. “Abnormal HDAC activity” is associated with aberrant gene or protein expression levels found in non pathological circumstances in an animal or a human. The expression level of genes from cells exposed to HDAC inhibitors differs in comparison with the level of expression found under physiological conditions in the absence of pathology or in cells with non pathological properties or behaviours. In one aspect the expression levels are increased or decreased at least about twice, preferably at least about four times, more preferably at least about ten times, most preferably at least about 100 times the amount of mRNA or proteins found in corresponding tissues samples from subjects who do not suffer from conditions associated with abnormal HDAC activity.

HDAC inhibitors may be suitable for use as therapeutic agents in mammals, including animals in veterinary medicine or humans, in need of treatment of diseases in which abnormal HDAC activity is implicated and HDAC inhibition is desirable. Such conditions include, but are not limited to, atherosclerosis, inflammatory bowel disease, host inflammatory or immune response, psoriasis and conditions associated with abnormal cell proliferation, such as cancer. In other aspects disorders of abnormal HDAC activity involves non-pathological circumstances such as apoptosis or cell cycle regulation which are not detected pathologically. Thus, given the clinical importance of HDAC inhibitors, a method to facilitate the detection of such useful therapeutic compounds from a chemical compound library of thousands is of significant value. Based on the surprising discovery that HDAC inhibition is associated with modified expression levels of any one or more genes selected from those disclosed in Table 1 and proteins encoded therefrom, it is contemplated herein that the expression levels of these genes or proteins can be used in a method to facilitate the identification of novel HDAC inhibitors by permitting the identification of compounds that are clearly not HDAC inhibitors. Compounds which are not HDAC inhibitors or therapeutically effective HDAC inhibitors can be identified as those compounds which do not cause an up- or down regulation of genes disclosed in Table 1 (e.g. mRNA levels and/or protein levels) compared to controls. These compounds may then be eliminated from the “list” of possible HDAC inhibitors and need not be tested further. Attention may then be focused on those compounds that actually produce expression levels of genes or proteins disclosed herein then further assayed using conventional methods to better characterize or to determine other interactions and pathways that are related to HDAC activity.

Based on this observation, it is clear that abnormal or modulated expression of genes selected from the group consisting of those disclosed in Table 1 or proteins encoded therefrom in a culture or subject compared to control levels indicates that HDACs are inhibited in said culture or subject. Such information can be used to biochemically characterize a particular cell type, including, for example, primary cultures of diseased cells from a subject or established cell lines as well as provide information regarding a disease state or other pathological condition in vivo and may thus provide useful information regarding appropriate clinical treatment options. It is further contemplated that mRNA, cDNA or proteins encoded thereby are also included in the invention disclosed herein and can be prepared or identified using public databases available one of skill in the art.

In one aspect, therefore, the present invention pertains to a method for screening for HDAC activity in vitro or in vivo comprising detecting expression levels of any one or more genes disclosed in Table 1 or proteins encoded therefrom in vitro or in vivo and comparing with expression levels of the same genes or proteins in appropriate controls. In this case, “controls” refers to samples, cells, cultures or in subjects (as the case may be) which may be used to compare expression levels of Table 1 genes or proteins encoded therefrom and will be familiar to one of skill in the art. A control sample from a subject is preferably taken from a matched individual, i.e., an individual of similar age, sex, or other general condition but who is not suspected of having a condition associated with abnormal HDAC activity. Alternatively, the control sample may be taken from the subject at a time when the subject is not suspected of having a conditions associated with abnormal HDAC activity.

In another aspect, the present invention relates to screening methods to detect HDAC inhibition in vitro comprising administering a compound to cells in vitro, assaying for expression levels of any one or more genes or proteins encoded by genes selected from Table 1 in said cells and in control cells to which no compound has been administered, and comparing expression levels of corresponding genes or proteins between said cells and control cells. The in vitro screening method may be performed using techniques familiar to one of skill in the art. For example, a compound may be screened in vitro using primary cells isolated from human or other mammalian subjects or using a variety of cancer cell lines such as H1299, A549, HCT116 or any other cells in which mRNA levels may be detectably expressed according to conventional methods. Such cell lines are available commercially, for example, from the ATCC (Manassas, Va.) and may be discerned by one of skill in the art without undue experimentation. Conventional reporter gene assays could be used in which the promoter region of a gene is placed upstream of a reporter gene, the construct transfected into a suitable cell (for example, a tumor cell line such as HeLa, CHO, or HEK293 or primary cells such as human diploid fibroblasts, endothelial or chondrocyte cells) and using conventional techniques, the cells assayed for compound activity that causes activation of the modifier promoter by detection of the expression of the reporter gene.

In further aspect, the invention pertains to a method for screening a compound for HDAC inhibitory activity in vivo comprising assaying for expression levels of any one or more genes or proteins encoded by genes selected from the group consisting of those disclosed in Table 1 in a subject The in vivo screening assay may be performed using conventional assays (both in vitro and in vivo). For example, protein activity levels, e.g., enzymatic activity levels, can be assayed in a test subject using a biological sample from the subject using conventional enzyme activity assays. Protein expression levels can be assayed in a test subject using conventional detection techniques described herein. Gene expression (e.g. mRNA or cDNA levels) may also be determined using methods familiar to one of skill in the art, including, for example, conventional Northern analysis or commercially available microarrays. Additionally, the effect of test compound inhibition of protein levels can be detected with an ELISA antibody-based assay or fluorescent labelling reaction assay. These techniques are readily available for high throughput screening and are familiar to one skilled in the art. Test subjects may include, but are not limited to, conventional experimental animal models such as mouse xenograft, orthotopic or metastatic tumor models as well as human patients in controlled, clinical studies familiar to one of skill in the art.

Specifically, in another aspect, the invention relates to a method to determine sensitivity of a cell to which a known HDAC inhibitor comprising administering-an HDAC inhibitor to cells in vitro, screening for differences in expression levels of genes disclosed in Table 1 or proteins encoded therefrom in said cells and in control cells to which no HDAC inhibitor has been administered, and comparing expression levels of corresponding genes or proteins between said cells and control cells. In an important aspect, cells which do not express the genes or proteins disclosed herein according to the invention exhibit a resistance to the HDAC inhibitor. Cells that may be analyzed according to this method include, but are not limited to, tumor cell lines as well as primary cultures of neoplastic or other cells obtained from a biological sample.

Levels of expression of genes or proteins encoded by genes disclosed in Table 1 can be assayed from a biological sample by any known method, including conventional techniques of RNA quantitation such as Northern blot analysis, quantitative PCR or DNA microarrays (e.g. as commercialised by Affymetrix, Santa Clara, Calif.). Procedures or techniques in which minute amounts of a specific piece of nucleic acid, RNA and/or DNA, are amplified as described in U.S. Pat. No. 4,683,195 issued Jul. 28, 1987. Generally, sequence information from the ends of the region of interest or beyond needs to be available, such that oligonucleotide primers can be designed; these primers will be identical or similar in sequence to opposite strands of the template to be amplified. The 5′ terminal nucleotides of the two primers may coincide with the ends of the amplified material. PCR can be used to amplify specific RNA sequences, specific DNA sequences from total genomic DNA, and cDNA transcribed from total cellular RNA, bacteriophage or plasmid sequences, etc. See generally Mullis et al., Cold Spring Harbor Symp. Quant. Biol., 51: 263 (1987); Erlich, ed., PCR Technology, (Stockton Press, N.Y., 1989). PCR is considered to be one, but not the only, example of a nucleic acid polymerase reaction method for amplifying a nucleic acid test sample comprising the use of a known nucleic acid as a primer and a nucleic acid polymerase to amplify or generate a specific piece of nucleic acid.

Expression levels of proteins may be detected using conventional techniques, for example, immunoassays or electrophoresis assays. For example, immunoassays can be used to detect or monitor levels of proteins disclosed in Table 1 in a biological sample using specific polyclonal or monoclonal antibodies in any standard immunoassay format. ELISA (enzyme linked immunosorbent assay) type assays as well as conventional Western blotting assays using monoclonal antibodies are also exemplary assays that can be utilized to make direct determination of levels of the marker protein. Antibodies specific to proteins selected from those disclosed in Table 1 are available commercially or can be produced in accordance with conventional methods.

Antibodies to the proteins disclosed herein may also be used diagnostically. For example, one could use these antibodies according to conventional methods to quantitate upregulation or downregulation of proteins in a subject; differences in expression levels of genes and proteins disclosed in Table 1 compared to a suitable controls could be indicative of various clinical forms or severity of any one or more pathological conditions associated with abnormal HDAC activity. Such information would also be useful to identify subsets of patients with any one or more of said conditions that may or may not respond to treatment with HDAC inhibitors. Similarly, it is contemplated herein that quantitating the expression level of genes or proteins disclosed herein in a subject would be useful for diagnosis and determining appropriate therapy; subjects with increased or decreased mRNA levels of any one or more of these proteins compared to appropriate control individuals would be considered suitable candidates for treatment with modulators as disclosed herein.

For example, described herein are methods for the production of antibodies capable of specifically recognizing one or more differentially expressed gene epitopes. Such antibodies may include, but are not limited to polyclonal antibodies, monoclonal antibodies (mAbs), humanized or chimeric antibodies, single chain antibodies, Fab fragments, F(ab′)₂ fragments, fragments produced by a Fab expression library, anti-idiotypic (anti-Id) antibodies, and epitope-binding fragments of any of the above.

For the production of antibodies to the polypeptides discussed herein, various host animals may be immunized by injection with the polypeptides, or a portion thereof. Such host animals may include, but are not limited to, rabbits, mice, goats, chicken, and rats. Various adjuvants may be used to increase the immunological response, depending on the host species, including, but not limited to, Freund's (complete and incomplete), mineral gels such as aluminum hydroxide, surface active substances such as lysolecithin, pluronic polyols, polyanions, peptides, oil emulsions, keyhole limpet hemocyanin, dinitrophenol, and potentially useful human adjuvants such as BCG (bacille Calmette-Guerin) and Corynebacterium parvum.

Polyclonal antibodies are heterogeneous populations of antibody molecules derived from the sera of animals immunized with an antigen, such as target gene product, or an antigenic functional derivative thereof. For the production of polyclonal antibodies, host animals such as those described above, may be immunized by injection with the polypeptides, or a portion thereof, supplemented with adjuvants as also described above.

Monoclonal antibodies, which are homogeneous populations of antibodies to a particular antigen, may be obtained by any technique which provides for the production of antibody molecules by continuous cell lines in culture. These include, but are not limited to the hybridoma technique of Kohler and Milstein, (1975, Nature 256:495-497; and U.S. Pat. No. 4,376,110), the human B-cell hybridoma technique (Kosbor et al., 1983, Immunology Today 4:72; Cole et al., 1983, Proc. Natl. Acad. Sci. USA 80:2026-2030), and the EBV-hybridoma technique (Cole et al., 1985, Monoclonal Antibodies And Cancer Therapy, Alan R. Liss, Inc., pp. 77-96). Such antibodies may be of any immunoglobulin class including IgG, IgM, IgE, IgA, IgD, IgY and any subclass thereof. The hybridoma producing the mAb of this invention may be cultivated in vitro or in vivo. Production of high titers of mAbs in vivo makes this the presently preferred method of production.

In addition, techniques developed for the production of “chimeric antibodies” (Morrison et al., 1984, Proc. Natl. Acad. Sci., 81:6851-6855; Neuberger et al., 1984, Nature, 312:604-608; Takeda et al., 1985, Nature, 314:452-454) by splicing the genes from a mouse antibody molecule of appropriate antigen specificity together with genes from a human antibody molecule of appropriate biological activity can be used. A chimeric antibody is a molecule in which different portions are derived from different animal species, such as those having a variable or hypervariable region derived from a murine mAb and a human immunoglobulin constant region.

Alternatively, techniques described for the production of single chain antibodies (U.S. Pat. No. 4,946,778; Bird, 1988, Science 242:423-426; Huston et al., 1988, Proc. Natl. Acad. Sci. USA 85:5879-5883; and Ward et al., 1989, Nature 334:544-546) can be adapted to produce differentially expressed gene-single chain antibodies. Single chain antibodies are formed by linking the heavy and light chain fragments of the Fv region via an amino acid bridge, resulting in a single chain polypeptide.

Most preferably, techniques useful for the production of “humanized antibodies” can be adapted to produce antibodies to the polypeptides, fragments, derivatives, and functional equivalents disclosed herein. Such techniques are disclosed in U.S. Pat. Nos. 5,932, 448; 5,693,762; 5,693,761; 5,585,089; 5,530,101; 5,910,771; 5,569,825; 5,625,126; 5,633,425; 5,789,650; 5,545,580; 5,661,016; and 5,770,429, the disclosures of all of which are incorporated by reference herein in their entirety.

Antibody fragments that recognize specific epitopes may be generated by known techniques. For example, such fragments include but are not limited to: the F(ab′)₂ fragments which can be produced by pepsin digestion of the antibody molecule and the Fab fragments which can be generated by reducing the disulfide bridges of the F(ab′)₂ fragments. Alternatively, Fab expression libraries may be constructed (Huse et al., 1989, Science, 246:1275-1281) to allow rapid and easy identification of monoclonal Fab fragments with the desired specificity.

Detection of the antibodies described herein may be achieved using standard ELISA, FACS analysis, and standard imaging techniques used in vitro or in vivo. Detection can be facilitated by coupling (i.e., physically linking) the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, (3-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I ³⁵S or ³H.

Particularly preferred, for ease of detection, is the sandwich assay, of which a number of variations exist, all of which are intended to be encompassed by the present invention. For example, in a typical forward assay, unlabeled antibody is immobilized on a solid substrate and the sample to be tested brought into contact with the bound molecule. After a suitable period of incubation, for a period of time sufficient to allow formation of an antibody-antigen binary complex, a second antibody, labeled with a reporter molecule capable of inducing a detectable signal, is added and incubated, allowing time sufficient for the formation of a ternary complex of antibody-antigen-labeled antibody. Any unreacted material is then washed away, and the presence of the antigen is determined by observation of a signal, or may be quantitated by comparing with a control sample containing known amounts of antigen. Variations on the forward assay include the simultaneous assay, in which both sample and antibody are added simultaneously to the bound antibody, or a reverse assay in which the labeled antibody and sample to be tested are first combined, incubated and added to the unlabeled surface bound antibody. These techniques are well known to those skilled in the art, and the possibility of minor variations will be readily apparent. As used herein, “sandwich assay” is intended to encompass all variations on the basic two-site technique. For the immunoassays of the present invention, the only limiting factor is that the labeled antibody be an antibody which is specific for the modifier polypeptide or fragments thereof.

The most commonly used reporter molecules are either enzymes, fluorophore- or radionucleotide-containing molecules. In the case of an enzyme immunoassay, an enzyme is conjugated to the second antibody, usually by means of glutaraldehyde or periodate. As will be readily recognized, however, a wide variety of different ligation techniques exist, which are well-known to the skilled artisan. Commonly used enzymes include horseradish peroxidase, glucose oxidase, beta-galactosidase and alkaline phosphatase, among others. The substrates to be used with the specific enzymes are generally chosen for the production, upon hydrolysis by the corresponding enzyme, of a detectable color change. For example, p-nitrophenyl phosphate is suitable for use with alkaline phosphatase conjugates; for peroxidase conjugates, 1,2-phenylenediamine or toluidine are commonly used. It is also possible to employ fluorogenic substrates, which yield a fluorescent product rather than the chromogenic substrates noted above. A solution containing the appropriate substrate is then added to the tertiary complex. The substrate reacts with the enzyme linked to the second antibody, giving a qualitative visual signal, which may be further quantitated, usually spectrophotometrically, to give an evaluation of the amount of polypeptide or polypeptide fragment of interest which is present in the serum sample.

Alternately, fluorescent compounds, such as fluorescein and rhodamine, may be chemically coupled to antibodies without altering their binding capacity. When activated by illumination with light of a particular wavelength, the fluorochrome-labeled antibody absorbs the light energy, inducing a state of excitability in the molecule, followed by emission of the light at a characteristic longer wavelength. The emission appears as a characteristic color visually detectable with a light microscope. Immunofluorescence and EIA techniques are both very well established assays and are particularly preferred for the present method. However, other reporter molecules, such as radioisotopes, chemiluminescent or bioluminescent molecules may also be employed. It will be readily apparent to those skilled in the art how to vary the procedure to suit the required use.

The present invention also provides a method for monitoring the therapeutic efficacy to a known HDAC inhibitor in a subject comprising detecting gene or protein expression levels in a subject before and after treatment with an HDAC inhibitor; and comparing expression levels in said subject of any one or more genes or proteins encoded by genes selected from those disclosed in Table 1. In one aspect, HDAC inhibiton is associated with a comparison of the differences in expression level if genes or proteins in said subject after treatment compared to levels before treatment. For example, circulating tumor epithelial cells, may be purified from the blood of patients undergoing HDAC inhibition treatment (i.e. biological samples obtained before and after treatment). mRNA are purified from these cells and the expression levels of the genes or proteins selected from those disclosed Table 1 are determined in the samples by RT-PCR according to conventional methods. A lack of expression of any one or more genes or proteins encoded by genes selected from Table 1 obtained from a patient to whom an HDAC inhibitor has been administered would be indicative of a lack of therapeutic efficacy to such HDAC inhibitor in said patient.

In a related aspect, the invention relates to a method to determine resistance of a subject to a known HDAC inhibitor comprising a). detecting expression levels of any one or more proteins selected from the group consisting of those disclosed in Table 1 in said subject before and after treatment with said HDAC inhibitor; and b). comparing expression levels in said subject wherein HDAC inhibition is associated with changes in expression levels in said subject after treatment compared to expression levels before treatment indicates resistance of said subject to the HDAC inhibitor. In a particular aspect the changes in expression level occur as downregulation of genes and/or upregulation of genes shown in Table 2. As is the case with all aspects of the invention disclosed herein, administration of HDAC inhibitors and analysis the genes in Table 1 expression levels in vitro and in vivo in order to determine resistance to an HDAC inhibitor may be performed according to a variety of conventional methods familiar to one of skill in the art and as in any of the techniques described herein.

It is contemplated herein that monitoring expression levels or activity and/or detecting gene expression (mRNA levels) of any one or more genes or proteins disclosed herein in a subject may be used as part of a clinical testing procedure, for example, to determine the efficacy of a given treatment regimen. Patients to whom a test substance has been administered would be clinically evaluated and patients in whom protein levels, activity and/or gene expression levels are different than desired (i.e. levels greater or less than levels in control patients or in patients in whom any one or more conditions has been sufficiently alleviated by clinical intervention) could be identified. Based on these data, the clinician could then adjust the dosage, administration regimen or type of therapeutic substance prescribed. Accordingly, the method of the present invention can be used to monitor the therapeutic efficacy of a compound and/or to find a therapeutically effective amount or regimen for the selected compound, thereby individually selecting and optimizing a therapy for a patient. Factors for consideration in this context include the particular condition being treated, the particular mammal being treated, the clinical condition of the individual patient, the site of delivery of the active compound, the particular type of the active compound, the method of administration, the scheduling of administration, and other factors known to medical practitioners. The therapeutically effective amount of an HDAC inhibitor to be administered will be governed by such considerations, and is the minimum amount necessary to prevent, ameliorate, or treat the disease. Such amount is preferably below the amount that is toxic to the host or renders the host significantly more susceptible to infections.

Thus in one aspect, In a another aspect the invention relates to a method of diagnosing a disease or subtype of a disease susceptible to treatment with HDAC inhibitory agents, which comprises measuring in cells of the subject which exhibits the disease expression levels of genes or proteins encoded by genes selected from the group consisting of those disclosed in Table 1. Said measuring preferably takes place ex vivo, i.e. outside of the body, for instance using tissue or blood which had previously been isolated from said subject.

In a related aspect, the invention relates to a method to treat, prevent or ameliorate pathological conditions associated with abnormal HDAC activity including, but not limited to proliferative disorders such as neoplasms, including solid and leukemia, comprising administering to a subject in need thereof a pharmaceutical composition comprising an effective amount of an HDAC inhibitor which can modulate expression of proteins selected from the group consisting of those disclosed in Table 1. In a particular aspect, additional modulators may be administered which can block or compete with interactions that may be involved in the regulation of the protein and necessary for, e.g., enzymatic or catalytic activity. Such modulators include antibodies directed to proteins or fragments thereof which are expressed upstream or downstream from the proteins disclosed herein. In certain particularly preferred embodiments, the pharmaceutical composition comprises antibodies that are highly selective for human forms of said proteins or portions thereof. Antibodies to said proteins may cause the aggregation of the proteins in a subject and thus inhibit or reduce protein activity, e.g. enzymatic activity in the pathway associated with abnormal HDAC activity. Such antibodies may also inhibit or decrease protein activity, for example, by interacting directly with active sites or by blocking access of substrates to active sites. Antibodies with inhibitory activity such as described herein can be produced and identified according to standard assays familiar to one of skill in the art.

The pharmaceutical compositions of the present invention may also comprise substances that inhibit the expression of disclosed modifiers at the nucleic acid level. Such molecules include ribozymes, antisense oligonucleotides, triple helix DNA, RNA aptamers, siRNA and/or double or single stranded RNA directed to an appropriate nucleotide sequence of nucleic acid encoding a modifier. Use of such molecules targeted against genes or proteins disclosed herein serve to inhibit HDAC activity. For example, antisense sequences may be used to inhibit genes which are shown to be downregulated upon inhibition with HDAC inhibitors. Alternatively, the inhibitory molecules may be used to inhibit regulatory sequences of genes upregulated or downregulated in conditions of abnormal HDAC activity. These inhibitory molecules may be created using conventional techniques by one of skill in the art without undue burden or experimentation. For example, modifications (e.g. inhibition) of gene expression can be obtained by designing antisense molecules, DNA or RNA, to the control regions of the genes encoding the polypeptides discussed herein, i.e. to promoters, enhancers, and introns. For example, oligonucleotides derived from the transcription initiation site, e.g., between positions −10 and +10 from the start site may be used. Notwithstanding, all regions of the gene may be used to design an antisense molecule in order to create those which gives strongest hybridization to the mRNA and such suitable antisense oligonucleotides may be produced and identified by standard assay procedures familiar to one of skill in the art.

Similarly, inhibition of the expression of gene expression may be achieved using “triple helix” base-pairing methodology. Triple helix pairing is useful because it causes inhibition of the ability of the double helix to open sufficiently for the binding of polymerases, transcription factors, or regulatory molecules. Recent therapeutic advances using triplex DNA have been described in the literature (Gee, J. E. et al. (1994) In: Huber, B. E. and B. I. Carr, Molecular and Immunologic Approaches, Futura Publishing Co., Mt. Kisco, N.Y.). These molecules may also be designed to block translation of mRNA by preventing the transcript from binding to ribosomes.

Ribozymes, enzymatic RNA molecules, may also be used to inhibit gene expression by catalyzing the specific cleavage of RNA. The mechanism of ribozyme action involves sequence-specific hybridization of the ribozyme molecule to complementary target RNA, followed by endonucleolytic cleavage. Examples which may be used include engineered “hammerhead” or “hairpin” motif ribozyme molecules that can be designed to specifically and efficiently catalyze endonucleolytic cleavage of gene sequences. Specific ribozyme cleavage sites within any potential RNA target are initially identified by scanning the target molecule for ribozyme cleavage sites which include the following sequences: GUA, GUU and GUC. Once identified, short RNA sequences of between 15 and 20 ribonucleotides corresponding to the region of the target gene containing the cleavage site may be evaluated for secondary structural features which may render the oligonucleotide inoperable. The suitability of candidate targets may also be evaluated by testing accessibility to hybridization with complementary oligonucleotides using ribonuclease protection assays.

Ribozyme methods include exposing a cell to ribozymes or inducing expression in a cell of such small RNA ribozyme molecules (Grassi and Marini, 1996, Annals of Medicine 28: 499-510; Gibson, 1996, Cancer and Metastasis Reviews 15: 287-299). Intracellular expression of hammerhead and hairpin ribozymes targeted to mRNA corresponding to at least one of the genes discussed herein can be utilized to inhibit protein encoded by the gene.

Ribozymes can either be delivered directly to cells, in the form of RNA oligonucleotides incorporating ribozyme sequences, or introduced into the cell as an expression vector encoding the desired ribozymal RNA. Ribozymes can be routinely expressed in vivo in sufficient number to be catalytically effective in cleaving mRNA, and thereby modifying mRNA abundance in a cell (Cotten et al., 1989 EMBO J. 8:3861-3866). In particular, a ribozyme coding DNA sequence, designed according to conventional, well known rules and synthesized, for example, by standard phosphoramidite chemistry, can be ligated into a restriction enzyme site in the anticodon stem and loop of a gene encoding a tRNA, which can then be transformed into and expressed in a cell of interest by methods routine in the art. Preferably, an inducible promoter (e.g., a glucocorticoid or a tetracycline response element) is also introduced into this construct so that ribozyme expression can be selectively controlled. For saturating use, a highly and constituently active promoter can be used. tDNA genes (i.e., genes encoding tRNAs) are useful in this application because of their small size, high rate of transcription, and ubiquitous expression in different kinds of tissues.

Therefore, ribozymes can be routinely designed to cleave virtually any mRNA sequence, and a cell can be routinely transformed with DNA coding for such ribozyme sequences such that a controllable and catalytically effective amount of the ribozyme is expressed. Accordingly the abundance of virtually any RNA species in a cell can be modified or perturbed.

Ribozyme sequences can be modified in essentially the same manner as described for antisense nucleotides, e.g., the ribozyme sequence can comprise a modified base moiety.

RNA aptamers can also be introduced into or expressed in a cell to modify RNA abundance or activity. RNA aptamers are specific RNA ligands for proteins, such as for Tat and Rev RNA (Good et al., 1997, Gene Therapy 4: 45-54) that can specifically inhibit their translation.

Gene specific inhibition of gene expression may also be achieved using conventional double or single stranded RNA technologies. A description of such technology may be found in WO 99/32619 which is hereby incorporated by reference in its entirety. In addition, siRNA technology has also proven useful as a means to inhibit gene expression (Cullen, B R Nat. Immunol. 2002 July;3(7):597-9; Martinez, J. et al. Cell 2002 Sep. 6;110(5):563).

Antisense molecules, triple helix DNA, RNA aptamers, dsRNA, ssRNA, siRNA and ribozymes of the present invention may be prepared by any method known in the art for the synthesis of nucleic acid molecules. These methods include techniques for chemically synthesizing oligonucleotides such as solid phase phosphoramidite chemical synthesis. Alternatively, RNA molecules may be generated by in vitro and in vivo transcription of DNA sequences encoding the genes of the polypeptides discussed herein. Such DNA sequences may be incorporated into a wide variety of vectors with suitable RNA polymerase promoters such as T7 or SP6. Alternatively, cDNA constructs that synthesize antisense RNA constitutively or inducibly can be introduced into cell lines, cells, or tissues.

Vectors may be introduced into cells or tissues by many available means, and may be used in vivo, in vitro or ex vivo. For ex vivo therapy, vectors may be introduced into stem cells taken from the patient and clonally propagated for autologous transplant back into that same patient. Delivery by transfection and by liposome injections may be achieved using methods that are well known in the art.

In addition, the genes and/or proteins identified herein can be used to identify other proteins, e.g. receptors, that are modified by the genes or proteins encoded by genes disclosed in Table 1 in tissues in vivo. Proteins thus identified can be used for drug screening to treat or further characterize pathological conditions associated with abnormal HDAC activity. To identify these genes, including those that are downstream of the genes or proteins disclosed herein, it is contemplated, for example, that one could use conventional methods to treat animals in conventional in vivo models of any one or more said pathological conditions with a specific inhibitor of any one or more genes or proteins encoded by genes disclosed in Table _, sacrifice the animals, remove tissue samples and isolate total RNA from the tissue and employ standard microarray assay technologies to identify expression levels that are altered relative to a control animal (animal to whom no inhibitor has been administered).

It is contemplated that one can also alter the function and/or expression of a gene or protein disclosed herein as a way to treat pathological conditions associated with abnormal HDAC activity by designing pharmaceutical compositions comprising, for example, antibodies to the proteins modulated by HDAC inhibitors as described herein and/or designing inhibitory antisense oligonucleotides, triple helix DNA, ribozymes, ssRNA, dsRNA, siRNA and RNA aptamers targeted to the genes for such proteins according to conventional methods disclosed herein.

The pharmaceutical compositions disclosed herein useful for treating, preventing and/or ameliorating pathological conditions associated with abnormal HDAC activity are to be administered to a patient at therapeutically effective doses. A therapeutically effective dose refers to that amount of the compound sufficient to result in the treatment, prevention, or amelioration of any one or more of said conditions and would be able to be determined by a clinician or other person possessing ordinary skill in the art.

Pharmaceutical compositions for use in accordance with the present invention may be formulated in a conventional manner using one or more physiologically acceptable carriers or excipients.

Thus, compounds and their physiologically acceptable salts and solvates may be formulated for administration by inhalation or insufflation (either through the mouth or the nose) or topical, oral, buccal, parenteral or rectal administration.

For oral administration, pharmaceutical compositions may take the form of, for example, tablets or capsules prepared by conventional means with pharmaceutically acceptable excipients such as binding agents (e.g., pregelatinized maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulphate). The tablets may be coated by methods well known in the art. Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol or fractionated vegetable oils); and preservatives (e.g., methyl or propyl-p-hydroxybenzoates or sorbic acid). The preparations may also contain buffer salts, flavoring, coloring and sweetening agents as appropriate.

Preparations for oral administration may be suitably formulated to give controlled release of the active compound.

For buccal administration the compositions may take the form of tablets or lozenges formulated in conventional manner.

For administration by inhalation, compounds for use according to the present invention are conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of, e.g., gelatin for use in an inhaler or insufflator may be formulated containing a powder mix of the compound and a suitable powder base such as lactose or starch.

Compounds may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle, e.g., sterile pyrogen-free water, before use.

Compounds may also be formulated in rectal compositions such as suppositories or retention enemas, e.g., containing conventional suppository bases such as cocoa butter or other glycerides.

Compounds may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

The compositions may, if desired, be presented in a pack or dispenser device which may contain one or more unit dosage forms containing the active ingredient. The pack may for example comprise metal or plastic foil, such as a blister pack. The pack or dispenser device may be accompanied by instructions for administration.

Pharmaceutical compositions suitable for use in the invention include compositions wherein the active ingredients are contained in an effective amount to achieve the intended purpose. The determination of an effective dose is well within the capability of those skilled in the art.

For any compound, the therapeutically effective dose can be estimated initially either in cell culture assays, e.g., of neoplastic cells, or in animal models, usually mice, rabbits, dogs, or pigs. The animal model may also be used to determine the appropriate concentration range and route of administration. A dose may be formulated in animal models to achieve a circulating plasma concentration range that includes the IC₅₀ (i.e., the concentration of the test compound that achieves a half-maximal inhibition of symptoms). Such information can then be used to determine useful doses and routes for administration in humans.

A therapeutically effective dose refers to that amount of active ingredient, for example, inhibitory compound, antisense oligonucleotides, triple helix DNA, ribozymes, RNA aptamer, siRNA or double or single stranded RNA designed to inhibit the expression of a gene encoding an modifier, antibodies to said modifiers or fragments thereof, useful to treat, prevent and/or ameliorate pathological conditions associated with abnormal HDAC activity. Therapeutic efficacy and toxicity may be determined by standard pharmaceutical procedures in cell cultures or experimental animals, e.g., ED50 (the dose therapeutically effective in 50% of the population) and LD50 (the dose lethal to 50% of the population). The dose ratio between toxic and therapeutic effects is the therapeutic index, and it can be expressed as the ratio, LD50/ED50. Pharmaceutical compositions that exhibit large therapeutic indices are preferred. The data obtained from cell culture assays and animal studies is used in formulating a range of dosage for human use. The dosage contained in such compositions is preferably within a range of circulating concentrations that include the ED50 with little or no toxicity. The dosage varies within this range depending upon the dosage form employed, sensitivity of the patient, and the route of administration.

The exact dosage will be determined by the practitioner, in light of factors related to the subject that requires treatment. Dosage and administration are adjusted to provide sufficient levels of the active moiety or to maintain the desired effect. Factors that may be taken into account include the severity of the disease state, general health of the subject, age, weight, and gender of the subject, diet, time and frequency of administration, drug combination(s), reaction sensitivities, and tolerance/response to therapy. Long-acting pharmaceutical compositions may be administered every 3 to 4 days, every week, or once every two weeks depending on half-life and clearance rate of the particular formulation.

Normal dosage amounts may vary from 0.1 to 100,000 micrograms, up to a total dose of about 1 g, depending upon the route of administration. Guidance as to particular dosages and methods of delivery is provided in the literature and generally available to practitioners in the art. Those skilled in the art will employ different formulations for nucleotides than for proteins or their inhibitors. Similarly, delivery of polynucleotides or polypeptides will be specific to particular cells, conditions, locations, etc. Pharmaceutical formulations suitable for oral administration of proteins are described, e.g., in U.S. Pat. Nos. 5,008,114; 5,505,962; 5,641,515; 5,681,811; 5,700,486; 5,766,633; 5,792,451; 5,853,748; 5,972,387; 5,976,569; and 6,051,561.

It is contemplated that the invention described herein is not limited to the particular methodology, protocols, and reagents described as these may vary. It is also to be understood that the terminology used herein is for the purpose of describing particular embodiments only, and is not intended to limit the scope of the present invention in any way.

Unless defined otherwise, all technical and scientific terms used herein have the same meanings as commonly understood by one of ordinary skill in the art to which this invention belongs. Although any methods and materials similar or equivalent to those described herein can be used in the practice or testing of the present invention, the preferred methods, devices and materials are now described. All publications mentioned herein are incorporated by reference for the purpose of describing and disclosing the materials and methodologies that are reported in the publication which might be used in connection with the invention. Persons skilled in the art will recognize, or be able to ascertain using no more than routine experimentation, many equivalents to the specific embodiments of the invention described herein. Such equivalents are intended to be encompassed by the claims.

The following examples further illustrate the present invention and are not intended to limit the invention.

### EXAMPLES

### EXAMPLE 1

Differential Expression in A549 Cells after LAQ824 Treatment

Data disclosed herein are the results of experiments using a hydroxamate compound which is a histone deacetylase inhibitor, N-Hydroxy-3-[4-[[(2-hydroxyethyl)[2-(1H-indol-3-yl)-ethyl]-amino]methyl]phenyl]-2E-2-propenamide, referred to herein as “Compound A” This and other generic compounds and their synthesis are discussed in detail in WO02/22577.

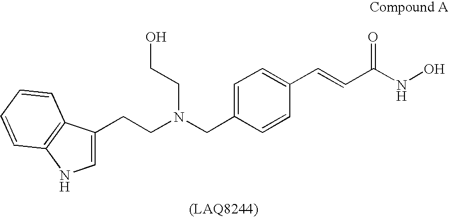

Compound A may be prepared according to the following synthesis: A solution of 3-(4-{[2-(1H-indol-3-yl)-ethylamino]-methyl}-phenyl)-(2E)-2-propenoic acid methyl ester (12.6 g, 37.7 mmol), (2-bromoethoxy)-tert-butyldimethylsilane (12.8 g, 53.6 mmol), (i-Pr)2NEt, (7.42 g, 57.4 mmol) in DMSO (100 mL) is heated to 50° C. After 8 hours the mixture is partitioned with CH2Cl2/H2O. The organic layer is dried (Na2SO4) and evaporated. The residue is chromatographed on silica gel to produce 3-[4-({[2-(tert-butyldimethylsilanyloxy)-ethyl]-[2-(1H-indol-3-yl)-ethyl]-amino}-methyl)-phenyl]-(2E)-2-propenoic acid methyl ester (13.1 g). Following the procedure described above for the preparation of Compound A, 3-[4-({[2-(tert-butyldimethylsilanyloxy)-ethyl]-[2-(1H-indol-3 -yl)-ethyl]-amino}-methyl)-phenyl]-(2E)-2-propenoic acid methyl ester (5.4 g, 11 mmol) is converted to N-hydroxy-3-[4-({[2-(tert-butyldimethylsilanyloxy)-ethyl]-[2-(1H-indol-3-yl)-ethyl]-amino}-methyl)-phenyl]-(2E)-2-propenamide (5.1 g,) and used without further purification. The hydroxamic acid (5.0 g, 13.3 mmol) is then dissolved in 95% TFA/H20 (59 mL) and heated to 40-50 ° C. for 4 hours. The mixture is evaporated and the residue purified by reverse phase HPLC to produce N-Hydroxy-3-[4-[[(2-hydroxyethyl)[2-(1H-indol-3-yl)-ethyl]-amino]methyl]phenyl]-2E-2-propenamide as the trifluoroacetate salt (2.19 g).

### Example 2

Cell Extract and Immunoblotting Analyses

A549 cells are cultured with different concentrations of LAQ824 or DMSO for 0.5, 2, 6, 12, 24 h. The cells are lysed by triple detergent lysis buffer (50 mM Tris.Cl, pH 8.0, 50 mM NaCl, 0.1% SDS, 1% NP40, 0.5% Sodium Deoxycholate, proteinase inhibitors) and whole cell lysis are extracted. Protein concentration is determined by using Bio-Rad reagent according to the manufacturer's protocol (30 μg of protein is used for analysis on SDS/PAGE gels). Gels are transferred to Nitrocellulose membranes (Bio-Rad Laboratories, Hercules, Calif.) and analyzed with specific histone antibodies. Membrane staining (Ponceau S solution, Sigma, St. louis, Mo.) is used as a protein-loading control. Each blocked membrane is incubated with respective primary antibodies against histone H3 isotype and modifications. The chemiluminescent histone signal is visualized using ECL Plus Western Blotting Detection System and Hyperfilm ECL autoradiography film (Amersham Pharmacia Biotech, Inc. Piscataway, N.J.).

The antibodies to the acetylated, methylated, and phosphorylated histones are purchased from Upstate Biotechnology (Lake Placid, N.Y.) and Abcam (Cambridge, Mass.). The following antibodies are used in this study (their catalog numbers are indicated): anti-acetylated histone H3 (H3, K9/K14, 06-599); anti-histone H3 (06-755); anti-H3 acetylated K9 (06-942); anti-H3 acetylated K14 (06-911); (07-030), anti-H3 trimethyl-K4 (Abcam, ab8580); anti-H3 monomethyl-K36 (Abcam, ab9048), anti-H3 dimethyl-K36 (07-369). anti-H3 monomethyl-K79 (Abcam, ab2886), anti-H3 dimethyl-K79 (07-366).

Using the antibodies described above, and upon consideration of the role of HDACs in determining chromatin structure, we additionally examined changes in post-translational modifications of histones in response to HDACi in asynchronized as well as cell cycle synchronized populations. Surprisingly, it was found that HDACi induced not only acetylation of histone H3, but also methylation of K4, K36 and K79 on H3. This suggests that acetylation and methylation of histones occur in a coordinated fashion and together establish epigenetic marks for specific genes or regions of chromatin.

In a related experiment, the mechanism of transcriptional regulation of RhoB by HDAC has been investigated by determining the effect of TPX inhibition on a series of RhoB promoter deletion constructs (Cohen ______ Interestingly, induction of expression from all of these deletions is similar following TPX treatment. To define the TPX responsive sequences, mutations are introduced into the CCAAT and the TATA boxes in the proximal region of the RhoB promoter. Only the promoter constructs harboring mutations in the inverted CCAAT box lost the ability to be induced following TPX treatment, indicating the involvement of this sequence in the repression of RhoB promoter activity by HDAC. Furthermore, mutations in this CCAAT box reduced the ability of HDAC1 to confer repression of RhoB promoter activity. In addition, chromatin immunoprecipitation assays reveal that TPX increased the level of chromatin acetylation in the TPX-responsive region of the RhoB promoter containing the inverted CCAAT box. Gel supershift assay indicate that NF—Y complex bind to this inverted CCAAT box in RhoB promoter in vitro, suggesting that NF—Y may be a component in the HDAC1/CCAAT complex. The global gene expression profiles of A549 cells following inhibition of HDAC with LAQ824 were determined by DNA microarray analysis. Among other genes, the NF—Y is one of the significantly up-regulated genes in response to LAQ824 treatment.

### Example 3

Microarray Analysis

The human lung carcinoma cell line A549 is obtained from the American Type Culture Collection (Rockville, Md.) and cultured in F-12K (ATCC, Manassas, Va.), 10% fetal bovine serum, and 1% penicillin/streptomycin (Life Technologies, Inc.). LAQ824 (Novartis) is added directly to the cell medium at a final concentration of 500 nM. Microarray analysis is performed essentially as described previously (Welsh, 2001). Total RNA is prepared from A549 cells treated with LAQ 824 or DMSO for 0.5, 2, 6, 12 and 24 h. Labelled cDNA is prepared and hybridized to Affymetrix U133 oligonucleotide GeneChip® (Affymetrix, Santa Clara, Calif.). The arrays were scanned on an Affymetrix GeneArray scanner and analyzed with GENECHIP Microarray Analysis Suite 5.0 software (MAS5) (Affymetrix). The data (raw data including image files and scanner files (Cel. Files) as well as the MAS5 normalized data are stored in the DEMON Database. The MAS5 normalized data are exported from the database and analyzed using GeneSpring software version 6.1. The genes are filtered for a minimal expression of 100 in at least one condition (the condition being defined as the average the value for the 3 samples corresponding to a given time point and a treatement) The gene expression changes (fold change) are calculated for each time point (t) as follow: (Gene expression level LAQ824 treated—(Average of the 3 replicates) Time (t))/(Gene expression level DMSO treated—(Average of the 3 replicates) Time (t)).

Any fold change <0 indicated a down regulation or reduction of the mRNA production, any fold change >0 indicated an up regulation or increase of mRNA production. Genes are filtered according to the fold change and the statistical significance of the changes (1-way-ANOVA test p<0.05)).

As shown in Table 1, further selection of the genes is made according to their fold change (at least 4-old for one of the time points considered) and their relationship to biological pathways of cell cycle and apoptosis shown in Table 2 including public available knowledge on the function of the genes.

TABLE 1

| | | | | |
|---|---|---|---|---|
| Description | Common | Genbank | Product | Affymetrix ID |

Genes down regulated
(expression upon HDAC inhibitor treatment < expression in control)

| | | | | |
|---|---|---|---|---|
| AMP-activated protein | ARK5 | NM_014840 | AMP-activated protein | 204589_at |
| kinase family member 5 | | | kinase family member 5 | |
| ANP32B acidic (leucine- | SSP29 | AV712577 | nuclear phospho protein | 201305_x_at |
| rich) nuclear phosphoprotein | | | | |
| 32 family, member B | | | | |
| AVEN apoptosis, caspase | Aven | NM_020371 | cell death regulator aven | 219366_at |
| activation inhibitor | | | | |
| B-cell CLL/lymphoma 3 | BCL3 | NM_005178 | B-cell CLL/lymphoma 3 | 204908_s_at |
| BCL2-associated | BAG-2 | AF095192 | BCL2-associated | 209406_at |
| athanogene 2 | | | athanogene 2 | |
| BCL2-like 1 | BCL2L1 | NM_001191 | BCL2-like 1, isoform 2 | 206665_s_at |
| BH3 interacting domain | BID | NM_001196 | BH3 interacting domain | 204493_at |
| death agonist | | | death agonist | |
| Cyclin A2 | CCNA | NM_001237 | cyclin A | 203418_at |
| DEC1-anti apoptosis | BHLHB2 | BG326045 | | 201169_s_at |
| Protein regulator of | PRC1 | NM_003981 | protein regulator of | 218009_s_at |
| cytokinesis 1 | | | cytokinesis 1 | |
| Serine/threonine kinase 6 | AIK | NM_003158 | serine/threonine kinase 6 | 208079_s_at |
| Tribbles homolog 3a | TRIB3 | NM_021158 | chromosome 20 open | 218145_at |
| | | | reading frame 97 | |
| Tumor necrosis factor | TNFRSF6 | X83493, | | 215719_x_at, |
| receptor superfamily, | | Z70519 | | 216252_x_at |
| member 6 | | | | |
| Tumor protein p53 (Li- | p53 | K03199 | tumor protein p53 | 201746_at |
| Fraumeni syndrome) | | | | 211300_s_at |
| Homo sapiens BAC | | AC004010 | | 222108_at |
| clone GS1-99H8 from 7, | | | | |
| complete sequence. | | | | |
| Hypothetical protein, | DKFZP586E011 | NM_015516 | hypothetical protein, | 218245_at |
| estradiol-induced | | | estradiol-induced | |
| Protein kinase domains | dJ1103G7.3 | NM_021158 | chromosome 20 open | 218145_at |
| containing protein similar to | | | reading frame 97 | |
| phosphoprotein C8FW | | | | |
| Tumor endothelial marker 6 | FLJ13732 | NM_022748 | tumor endothelial marker 6 | 217853_at |
| SLC7A11: solute carrier | ESTs | AA488687 | | 217678_at |
| family 7, (cationic amino acid | | | | |
| transporter, y+ system) | | | | |
| member 11 | | | | |
| NRP1 neuropilin 1 | NRP1 | BE620457 | | 212298_at |
| VEGF165R | | | | |
| Solute carrier family 7, | SLC7A11 | AB040875 | cystine/glutamate exchanger | 218245_at |
| (cationic amino acid | | | | |
| transporter, y+ system) | | | | |
| member 11 | | | | |
| Inhibitor of DNA binding 3, | HEIR-1V | NM_002167 | inhibitor of DNA binding 3, | 207826_s_at |
| dominant negative helix- | | | dominant negative helix- | |
| loop-helix protein | | | loop-helix protein | |
| Cytochrome P450, subfamily | CP24, | NM_000782 | cytochrome P450, subfamily | 206504_at |
| XXIV (vitamin D 24- | P450- | | XXIV precursor | |
| hydroxylase) | CC24 | | | |
| Tripartite motif-containing 16 | EBBP | NM_006470 | tripartite motif-containing 16 | 204341_at |
| CCAAT/enhancer binding | CELF, | NM_005195 | CCAAT/enhancer binding | 203973_s_at |
| protein (C/EBP), delta | CRP3 | | protein (C/EBP), delta | |

Genes up regulated
(expression upon HDAC inhibitor treatment > expression in control)

| | | | | |
|---|---|---|---|---|
| BAI1-associated protein 2 | BAP2, | AB017120 | BAI1-associated protein 2, | 205293_x_at |
| | IRSp53 | | isoform 1 | |
| BAI1-associated protein 3 | BAP3, | AB018277 | BAI1-associated protein 3 | 204874_x_at |
| | | | | 216356_x_at |
| BCL2-interacting killer | BP4, NBK, | NM_001197 | BCL2-interacting killer | 205780_at |
| (apoptosis-inducing) | BIP1 | | | |
| Cyclin-dependent kinase | INK4D, | U20498 | cyclin-dependent kinase | 210240_s_at |
| inhibitor 2D (p19, inhibits | p19- | | inhibitor 2D | |
| CDK4) | INK4D | | | |
| Diphtheria toxin receptor | DTR | NM_001945 | (heparin-binding epidermal | 203821_at |
| | | | growth factor-like growth | |
| | | | factor) | |
| Early growth response 1 | TIS8, | NM_001964 | early growth response 1 | 201694_s_at |
| | NGFI-A, | | | |
| | KROX-24, | | | |
| | ZIF-268 | | | |
| GMCL germ cell-less | GMCL | NM_022471 | hypothetical protein | 218458_at |
| homolog (Drosophila) | | | FLJ13057 similar to germ | |
| | | | cell-less | |
| Katanin p80 (WD40- | KAT | NM_005886 | katanin p80 subunit B 1 | 203163_at |
| containing) subunit B 1 | | | | |
| NDRG4 | NDRG4 | AV724216. | member of the N-myc | 209159_s_at |
| | | | downregulated gene family | |
| | | | which belongs to the | |
| | | | alpha/beta hydrolase | |
| | | | superfamily | |
| NACHT, leucine rich repeat | PP1044 | NM_021730 | hypothetical protein PP1044 | 218380_at |
| and PYD containing 1 | | | | |
| Paternally expressed Gene | PEG10 | BE858180 | | 212094_at, |
| 10 | | | | 212092_at |
| Secreted frizzled-related | FRP-1, | AF017987 | secreted frizzled-related | 202037_s_at |
| protein 1 | SARP2 | | protein 1 | 202036_s_at |
| SKAP55 protein | WUGSC: | AC003999 | | 216899_s_at |
| | H_DJ1139P01.1 | | | |
| Insulin induced gene 1 | INSIG1 | NM_005542 | insulin induced gene 1 | 201627_s_at |
| Insulin induced gene 2 | INSIG1 | BE300521 | | 201626_at |
| Insulin induced gene 3 | INSIG1 | BE300521 | | 201625_s_at |
| Protein tyrosine | PTP1B, | NM_002827 | protein tyrosine | 202716_at |
| phosphatase, non-receptor | PTP-1B | | phosphatase, non-receptor | |
| type | | | type 1 | |
| Niemann-Pick disease, type | NPC | NM_000271 | Niemann-Pick disease, type | 202679_at |
| C1 | | | C1 | |
| Sialidase 1 (lysosomal | NEU1 | U84246 | lysosomal sialidase | 208926_at |
| sialidase) | | | | |
| Acetylserotonin O- | ASTML, | BC002508 | acetylserotonin O- | 209394_at |
| methyltransferase-like | ASMTLX | | methyltransferase-like | |
| Solute carrier family 9 | EBP50, | NM_004252 | solute carrier family 9 | 201349_at |
| (sodium/hydrogen | NHERF | | (sodium/hydrogen | |
| exchanger), isoform 3 | | | exchanger), isoform 3 | |
| regulatory factor | | | regulatory factor 1 | |
| acetylserotonin O- | ASMTL | AA669799 | acetylserotonin O- | 36553_at |
| methyltransferase-like | | | methyltransferase-like | |
| acetylserotonin O- | ASMTL | Y15521 | acetylserotonin O- | 36554_at |
| methyltransferase-like | | | methyltransferase-like | |
| Interferon, gamma-inducible | IFI30 | NM_006332 | interferon, gamma-inducible | 201422_at |
| protein 30 | | | protein 30 | |
| Stanniocalcin 1 | STC1 | AI300520 | | 204595_s_at |
| Stanniocalcin 2 | STC1 | AK000345 | | 214079_at |
| Aldolase C, fructose- | ALDOC | NM_005165 | aldolase C, fructose- | 202022_at |
| bisphosphate | | | bisphosphate | |
| Myeloid leukemia factor 1 | MLF1 | NM_022443 | myeloid leukemia factor 1 | 204784_s_at |
| Short-chain alcohol | HEP27 | NM_005794 | short-chain alcohol | 206463_s_at |
| dehydrogenase family | | | dehydrogenase family | |
| member | | | member | |
| Hemoglobin, alpha 1 | HBA1 | NM_000558 | alpha 1 globin | 204018_x_at |
| Hemoglobin, alpha 2 | HBA2 | BC005931 | alpha 2 globin | 211745_x_at |
| Hemoglobin, alpha 1 | HBA1 | AF349571 | alpha 1 globin | 211699_x_at |
| C/EBP-induced protein | LOC81558 | NM_030802 | C/EBP-induced protein | 221249_s_at |
| Hemoglobin, alpha 2 | HBA2 | T50399 | | 214414_x_at |
| Stanniocalcin 1 | STC | NM_003155 | stanniocalcin 1 | 204597_x_at |
| Stanniocalcin 1 | STC | U46768 | stanniocalcin 1 | 204596_s_at |
| Transmembrane 7 | TM7SF2 | AF096304 | putative sterol reductase | 210130_s_at |
| superfamily member 2 | | | SR-1 | |
| Hemoglobin, alpha 2 | HBA2 | AF105974 | alpha one globin | 209458_x_at |
| Solute carrier family 17 | BNPI, | NM_020309 | solute carrier family 17, | 204230_s_at |
| (sodium-dependent inorganic | VGLUT1 | | member 7 | |
| phosphate cotransporter), | | | | |
| member 7 | | | | |
| Interferon regulatory factor 7 | IRF7 | NM_004030 | interferon regulatory factor 7 | 208436_s_at |
| | | | isoform a | |

TABLE 2
Fold changes for genes shown in Table 1

| | | Fold changes | | | | | | |
|---|---|---|---|---|---|---|---|---|
| Description | Common | 2 h | 6 h | 12 h | 24 h | Genbank | Product | Systematic |
| BCL2-like 1 | BCL2L1 | −1.2 | −4.2 | −7.6 | −2.0 | NM_001191 | BCL2-like 1, | 206665_s_at |
| | | | | | | | isoform 2 | |
| Tumor necrosis | TNFRSF6 | −1.3 | −3.2 | −6.3 | −4.8 | X83493 | | 215719_x_at |
| factor receptor | | | | | | | | |
| superfamily, | | | | | | | | |
| member 6 | | | | | | | | |
| B-cell | BCL3 | −1.9 | −10.5 | −5.5 | −1.8 | NM_005178 | B-cell | 204908_s_at |
| CLL/lymphoma 3 | | | | | | | CLL/lymphoma 3 | |
| ANP32B acidic | SSP29 | −1.3 | −1.6 | −5.1 | −7.3 | AV712577 | | 201305_x_at |
| (leucine-rich) | | | | | | | | |
| nuclear | | | | | | | | |
| phosphoprotein | | | | | | | | |
| 32 family, | | | | | | | | |
| member B | | | | | | | | |
| Tumor necrosis | TNFRSF6 | −1.3 | −2.9 | −4.9 | −5.6 | Z70519 | FAS soluble | 216252_x_at |
| factor receptor | | | | | | | protein | |
| superfamily, | | | | | | | | |
| member 6 | | | | | | | | |
| DEC1-anti | BHLHB2 | −3.2 | −4.5 | −4.8 | −1.9 | BG326045 | | 201169_s_at |
| apoptosis | | | | | | | | |
| AMP-activated | ARK5 | −1.1 | −4.3 | −4.6 | −1.9 | NM_014840 | KIAA0537 gene | 204589_at |
| protein kinase | | | | | | | product | |
| family member 5 | | | | | | | | |
| Tumor protein | P53, p53 | 1.1 | −1.9 | −4.4 | −7.1 | NM_000546 | tumor protein | 201746_at |
| p53 (Li-Fraumeni | | | | | | | p53 | |
| syndrome) | | | | | | | | |
| Tribbles homolog | TRIB3 | −1.3 | −4.6 | −4.0 | −10.1 | NM_021158 | chromosome | 218145_at |
| 3a | | | | | | | 20 open | |
| | | | | | | | reading frame | |
| | | | | | | | 97 | |
| Tumor protein | P53, p53 | 1.1 | −1.6 | −3.7 | −7.3 | K03199 | tumor protein | 211300_s_at |
| p53 (Li-Fraumeni | | | | | | | p53 | |
| syndrome) | | | | | | | | |
| BH3 interacting | BID | 1.1 | −1.9 | −3.6 | −4.6 | NM_001196 | BH3 interacting | 204493_at |
| domain death | | | | | | | domain death | |
| agonist | | | | | | | agonist | |
| BCL2-associated | BAG-2 | −1.4 | −2.4 | −3.3 | −4.4 | AF095192 | BCL2- | 209406_at |
| athanogene 2 | | | | | | | associated | |
| | | | | | | | athanogene 2 | |
| AVEN | Aven | −1.2 | −1.5 | −2.6 | −8.2 | NM_020371 | cell death | 219366_at |
| apoptosis, | | | | | | | regulator aven | |
| caspase | | | | | | | | |
| activation | | | | | | | | |
| inhibitor | | | | | | | | |
| Protein regulator | PRC1 | −1.2 | −1.9 | −2.5 | −5.1 | NM_003981 | protein | 218009_s_at |
| of cytokinesis 1 | | | | | | | regulator of | |
| | | | | | | | cytokinesis 1 | |
| Serine/threonine | AIK | 1.0 | −2.0 | −2.4 | −8.3 | NM_003158 | | 208079_s_at |
| kinase 6 | | | | | | | | |
| Cyclin A2 | CCN1, CCNA | −1.3 | −2.3 | −2.3 | −13.3 | NM_001237 | cyclin A | 203418_at |
| SKAP55 protein | WUGSC: H_DJ1139P01.1 | 1.0 | 1.6 | 1.8 | 4.0 | AC003999 | | 216899_s_at |
| Paternally | PEG10 | 1.1 | 1.9 | 3.5 | 6.5 | BE858180 | | 212094_at |
| expressed Gene | | | | | | | | |
| 10 | | | | | | | | |
| Paternally | PEG10 | 1.1 | 2.0 | 3.8 | 7.0 | BE858180 | | 212092_at |
| expressed Gene | | | | | | | | |
| 10 | | | | | | | | |
| GMCL germ cell- | GMCL | 1.7 | 4.1 | 3.8 | 2.3 | NM_022471 | hypothetical | 218458_at |
| less homolog | | | | | | | protein | |
| (Drosophila) | | | | | | | FLJ13057 | |
| | | | | | | | similar to germ | |
| | | | | | | | cell-less | |
| Secreted frizzled- | FRP, FRP1, | 1.1 | 1.8 | 3.9 | 4.4 | NM_003012 | secreted | 202037_s_at |
| related protein 1 | FrzA, FRP-1, | | | | | | frizzled-related | |
| | SARP2 | | | | | | protein 1 | |
| Secreted frizzled- | FRP, FRP1, | 1.1 | 1.6 | 3.9 | 4.5 | AF017987 | secreted | 202036_s_at |
| related protein 1 | FrzA, FRP-1, | | | | | | frizzled-related | |
| | SARP2 | | | | | | protein 1 | |
| Katanin p80 | KAT | 1.5 | 2.9 | 4.3 | 2.9 | NM_005886 | katanin p80 | 203163_at |
| (WD40- | | | | | | | subunit B 1 | |
| containing) | | | | | | | | |
| subunit B 1 | | | | | | | | |
| Cyclin-dependent | INK4D, p19- | 1.9 | 4.9 | 4.8 | 2.0 | U20498 | cyclin- | 210240_s_at |
| kinase inhibitor | INK4D | | | | | | dependent | |
| 2D (p19, inhibits | | | | | | | kinase inhibitor | |
| CDK4) | | | | | | | 2D | |
| BAI1-associated | BAP2, | 1.4 | 6.2 | 6.8 | 3.6 | AB017120 | BAI1- | 205293_x_at |
| protein 2 | IRSp53 | | | | | | associated | |
| | | | | | | | protein 2, | |
| | | | | | | | isoform 1 | |
| member of the N- | NDRG4 | 1.2 | 2.8 | 7.3 | 4.5 | AV724216 | | 209159_s_at |
| myc | | | | | | | | |
| downregulated | | | | | | | | |
| gene family | | | | | | | | |
| which belongs to | | | | | | | | |
| the alpha/beta | | | | | | | | |
| hydrolase | | | | | | | | |
| superfamily. | | | | | | | | |
| Early growth | TIS8, NGFI-A, | 1.4 | 3.6 | 11.2 | 4.6 | NM_001964 | early growth | 201694_s_at |
| response 1 | KROX-24, | | | | | | response 1 | |
| | ZIF-268 | | | | | | | |
| NACHT, leucine | PP1044 | 2.8 | 7.6 | 11.3 | 22.7 | NM_021730 | hypothetical | 218380_at |
| rich repeat and | | | | | | | protein PP1044 | |
| PYD containing 1 | | | | | | | | |
| BCL2-interacting | BP4, NBK, | 2.0 | 6.3 | 12.0 | 15.5 | NM_001197 | BCL2- | 205780_at |
| killer (apoptosis- | BIP1 | | | | | | interacting killer | |
| inducing) | | | | | | | | |
| Diphtheria toxin | DTR | 9.8 | 8.5 | 13.1 | 3.2 | NM_001945 | diphtheria toxin | 203821_at |
| receptor | | | | | | | receptor | |
| (heparin-binding | | | | | | | (heparin- | |
| epidermal growth | | | | | | | binding | |
| factor-like growth | | | | | | | epidermal | |
| factor) | | | | | | | growth factor- | |
| | | | | | | | like growth | |
| | | | | | | | factor) | |
| BAI1-associated | BAP3, | 1.0 | 5.1 | 15.9 | 10.9 | NM_003933 | BAI1- | 204874_x_at |
| protein 3 | KIAA0734 | | | | | | associated | |
| | | | | | | | protein 3 | |
| BAI1-associated | BAP3, | 2.3 | 11.1 | 27.9 | 19.2 | AB018277 | BAI1- | 216356_x_at |
| protein 3 | KIAA0734 | | | | | | associated | |
| | | | | | | | protein 3 | |

The description of genes disclosed in Table 2 are summarized as follows:

### Cell Cycle Genes

SKAP55, SCAP1 src family associated phosphoprotein 1 [Homo sapiens]

Gene name and aliases: SCAP1, SKAP55

Gene description: The protein encoded by this gene belongs to the src family kinases. It is a cytoplasmic protein which is preferentially expressed in T-lymphocytes where it interacts with the protein-tyrosine kinase p59fyn. The presence of a PH domain and a SH3 domain suggests that this encoded protein is capable of interacting with several other intracellular proteins.
SKAP-55 regulates integrin-mediated adhesion and conjugate formation between T cells and antigen-presenting cells
observation that adapter protein SKAP55 formed homodimers through its SH3 domain and SK region
SKAP55 coupled with CD45 positively regulates T-cell receptor-mediated gene transcription.

FRP, Secreted frizzled-related protein 1

Name and Alias: FRP; FRP1; FrzA; FRP-1; SARP2

Gene description: Secreted frizzled-related protein 1 (SFRP1) is a member of the SFRP family that contains a cysteine-rich domain homologous to the putative Wnt-binding site of Frizzled proteins. SFRPs act as soluble modulators of Wnt signaling. It is similar to the Wnt receptor and acts as antagonist.
SFRP1 is involved in the apoptosis regulation: protect fibroblast from ceramide induced apoptosis. Overexpression and knock-down by siRNA affect the expression level of p53, caspase 3 and 9 and BIK
SFRP1 expression is directed by CREB (test on promoter screen and ChIP)
SFRP1 loss of expression has been link to poor prognosis and high grade tumors

p19-INK4D, CDKiN2D cyclin-dependent kinase inhibitor 2D (p19, inhibits CDK4) [Homo sapiens]

Gene name and aliases: CDKN2D, p19; INK4D; p19-INK4D

Description: The protein encoded by this gene is a member of the fNK4 family of cyclin-dependent kinase inhibitors. This protein has been shown to form a stable complex with CDK4 or CDK6, and prevent the activation of the CDK kinases, thus function as a cell growth regulator that controls cell cycle G1 progression. The abundance of the transcript of this gene was found to oscillate in a cell-cycle dependent manner with the lowest expression at mid G1 and a maximal expression during S phase. The negative regulation of the cell cycle involved in this protein was shown to participate in repressing neuronal proliferation, as well as spermatogenesis. Two alternatively spliced variants of this gene, which encode an identical protein, have been reported.
p19(INK4d) in addition to p21 (WAF1/Cip1) is an important molecular target of HDAC inhibitors inducing growth arrest
Deletions in the CDKN2D locus and hypermethylation in the CDKN2D promoter region plays a role in ovarian granulosa cell tumorogenesis.
The human p19(INK4d) gene is under the control of TATA-less promoter and the Sp1 binding site is involved in the transcription.

DTR diphtheria toxin receptor (heparin-binding epidermal growth factor-like growth factor) [Homo sapiens]

Gene name and aliases: DTR, DTS; HBEGF; HEGFL

Gene description: diphtheria toxin receptor (heparin-binding epidermal growth factor-like growth factor), a member of the ErbB receptor ligand family, exists in distinct molecular forms with disparate biological activities. HB-EGF is a multifunctional member of the EGF-like growth factor family and elicits diverse effects in cells. HB-EGF is initially synthesized as a membrane-anchored precursor, proHB-EGF, which can participate in juxtacrine interactions with the EGFR and/or the ErbB4 receptor tyrosine kinase expressed on adjacent cells, leading to receptor activation and increased cell-cell adhesion. Membrane-anchored HB-EGF also serves as the source of soluble HB-EGF, which is released after a regulated, metalloproteinase cleavage step. Soluble HB-EGF mediates paracrine and autocrine activation of the EGFR and ErbB4 and thereby promotes survival, proliferation, and migration in different cell types.
HB-EGF is a potent inducer of tumor growth and angiogenesis
HB-EGF might be a more important tumor growth regulator of malignant fibrous histiocytoma through autocrine or paracrine pathways, when compared with betacellulin.
Transgenic expression of HB-EGF accelerates the proliferation of hepatocytes after partial hepatectomy
HB-EGF mRNA and protein levels in gastric cancers were elevated, compared with normal gastric tissues, especially in the intestinal type.

NDRG4 NDRG family member 4 [Homo sapiens]

Gene name and aliases: NDRG4, SMAP-8; KIAA1180; MGC19632

Gene description: NDRG family member 4, this gene is a member of the N-myc downregulated gene family which belongs to the alpha/beta hydrolase superfamily. The protein encoded by this gene is a cytoplasmic protein that may be involved in the regulation of mitogenic signalling in vascular smooth muscles cells.
map8 is involved in the regulation of mitogenic signalling in vascular smooth muscle cells, possibly in response to a homocysteine-induced injury [SMAP8]
Cloning and expression of the gene; specifically expressed in brain and heart

EGR1 early growth response 1 [Homo sapiens]

Gene name and aliases: EGR1, TIS8; AT225; NGFI-A; ZNF225; KROX-24; ZIF-268

Gene description: early growth response 1. The protein encoded by this gene belongs to the EGR family of C2H2-type zinc-finger proteins. It is a nuclear protein and functions as a transcriptional regulator. The products of target genes it activates are required for differentiation and mitogenesis. Expression of early growth response (Egr)-1, a transcriptional factor implicated in growth regulation, is suppressed in several malignant tumors. Studies suggest this is a cancer suppressor gene.
Egr-1 may display tumor-suppressing activity and offers a potential target for uterine leiomyoma management.
study provides the first evidence of the crucial role of Egr-1 in microvascular endothelial cell growth, neovascularization, tumor angiogenesis and tumor growth
Egr1 controls p21Cip1 expression by directly interacting with a specific sequence on its gene promoter.

KATNB1 katanin p80 (WD repeat containing) subunit B1 [Homo sapiens]

Gene name and aliases: KATNB1, KAT

Gene description: katanin p80 (WD repeat containing) subunit B 1. Microtubules, polymers of alpha and beta tubulin subunits, form the mitotic spindle of a dividing cell and help to organize membranous organelles during interphase. Katanin is a heterodimer that consists of a 60 kDa ATPase (p60 subunit A 1) and an 80 kDa accessory protein (p80 subunit B 1). The p60 subunit acts to sever and disassemble microtubules, while the p80 subunit targets the enzyme to the centrosome. Katanin is a member of the AAA family of ATPases.
PF15p is the chlamydomonas homologue of the Katanin p80 subunit and is required for assembly of flagellar central microtubules.
The Arabidopsis lue1 mutant defines a katanin p60 ortholog involved in hormonal control of microtubule orientation during cell growth.
MEI-1/MEI-2 katanin-like microtubule severing activity is required for Caenorhabditis elegans meiosis.
Two domains of p80 katanin regulate microtubule severing and spindle pole targeting by p60 katanin.
Katanin, a microtubule-severing protein, is a novel AAA ATPase that targets to the centrosome using a WD40-containing subunit.

AIK, Aurora-A, STK6 serine/threonine kinase 6 [Homo sapiens]

Gene name and aliases: STK6, AIK; ARK1; AURA; BTAK; STK15; MGC34538

Gene description: serine/threonine kinase 6. The protein encoded by this gene is a cell cycle-regulated kinase that appears to be involved in microtubule formation and/or stabilization at the spindle pole during chromosome segregation. The encoded protein is found at the centrosome in interphase cells and at the spindle poles in mitosis. Aurora kinases have recently taken centre stage in the regulation of key cell cycle processes. Aurora A is emerging as a critical regulator of centrosome and spindle function. In several common human tumors, Aurora-A is overexpressed, and deregulation of this kinase was shown to result in mitotic defects and aneuploidy. This gene may play a role in tumor development and progression.
BRCA1 phosphorylation by Aurora-A plays a role in G(2) to M transition of cell cycle
phosphorylation of CENP-A on Ser-7 by Aurora-A in prophase is essential for kinetochore function.
Aurora A protein kinase has a role in G2/M progression
results show that STK15 is overexpressed in pancreatic tumors and carcinoma cell lines and suggest that overexpression of STK15 may play a role in pancreatic carcinogenesis
elevated Aurora-A expression causes resistance to apoptosis in human cancer cell line
Aurora-A binds to TPX2, a prominent component of the spindle apparatus
Cell-cycle-dependent regulation of human aurora A transcription is mediated by periodic repression of E4TF1

CCNA2 cyclin A2 [Homo sapiens]

Gene name and aliases: CCNA2, CCN1; CCNA

Gene description: cyclin A2. The protein encoded by this gene belongs to the highly conserved cyclin family, whose members are characterized by a dramatic periodicity in protein abundance through the cell cycle. Cyclins function as regulators of CDK kinases. Different cyclins exhibit distinct expression and degradation patterns which contribute to the temporal coordination of each mitotic event. In contrast to cyclin A1, which is present only in germ cells, this cyclin is expressed in all tissues tested. This cyclin binds and activates CDC2 or CDK2 kinases, and thus promotes both cell cycle G1/S and G2/M transitions.
cyclin A is important regulator for cell cycle as well as for apoptosis
found that cyclin A and cyclin E are able to regulate both nuclear and cytoplasmic events because they both shuttle between the nucleus and the cytoplasm
Centrosome overduplication, increased ploidy and transformation in cells
expressing endoplasmic reticulum-associated cyclin A2
p53-dependent G2 arrest associated with a decrease in cyclins A2 and B1 levels in a human carcinoma cell line.
E2F1 and cyclin A2 may be induced by c-Myc to mediate the onset of mammary cancer.

PRC1 protein regulator of cytokinesis 1 [Homo sapiens]

Gene name and aliases: PRC1, MGC1671; MGC3669

Gene description: protein regulator of cytokinesis 1. This gene encodes a protein that is involved in cytokinesis. The encoded protein is at high level during S and G2/M and drop dramatically after cell exit mitosis and enter G1. It is located in the nucleus during interphase, and becomes associated with mitotic spindles in a highly dynamic manner during mitosis, and localizes to the cell mid-body during cytokinesis. This protein has been shown to be a substrate of several cyclin-dependent kinases (CDKs). At least three alternatively spliced transcript variants encoding distinct isoforms have been observed.
contributes to the correct formation of the spindle during the metaphase
PRC1 is a microtubule-associated protein required to maintain the spindle midzone, and that distinct functions are associated with modular elements of the primary sequence.

### Apoptosis Related Genes

GCL germ cell-less homolog 1 (Drosophila) [Homo sapiens]

Gene name and aliases: GCL; GMCL1; FLJ13057

Gene description: germ cell-less homolog 1 (Drosophila). This gene encodes a nuclear envelope protein that appears to be involved in spermatogenesis, either directly or by influencing genes that play a more direct role in the process. This multi-exon locus is the homolog of the mouse and drosophila germ cell-less gene but the human genome also contains a single-exon locus on chromosome 5 that contains an open reading frame capable of encoding a highly-related protein.
Germ cell-less protein is not essential for the chromosomal events of meiosis but might be involved in later aspects of spermatogenesis.
mGCL-1 is a factor modulating MDM2-p53 axis by enhanced degradation of MDM2
GCL binds directly to emerin, and the emerin-repressor complexes might be regulated by barrier to autointegration factor

BAIAP2 BAI1-associated protein 2 [Homo sapiens]

Gene name and aliases: BAIAP2, BAP2; IRSP53

Gene description: BAI1-associated protein 2. The protein encoded by this gene has been identified as a brain-specific angiogenesis inhibitor (BAII)-binding protein. This interaction at the cytoplasmic membrane is crucial to the function of this protein, which may be involved in neuronal growth-cone guidance. This protein functions as an insulin receptor tyrosine kinase substrate and suggests a role for insulin in the central nervous system. This protein has also been identified as interacting with the dentatorubral-pallidoluysian atrophy gene, which is associated with an autosomal dominant neurodegenerative disease. It also associates with a downstream effector of Rho small G proteins, which is associated with the formation of stress fibers and cytokinesis. Alternative splicing of the 3′-end of this gene results in three products of undetermined function.
IRSp53, when activated by small GTPases, participates in F-actin reorganization not only in an SH3-dependent manner but also in a manner dependent on the activity of the IRSp53/MIM homology domain
LIN7B is a partner of IRSp53 anchoring the actin-based membrane cytoskeleton at cell-cell contacts.

PP1044, NACHT, NAPL1

Name and Alias: CARD7; DEFCAP; PP1044; KIAA0926; DEFCAP-L/S; DKFZp586O1822

Gene description: NACHT is a member of the Ced-4 family of apoptosis proteins. Ced-family members contain a caspase recruitment domain (CARD) and are known to be key mediators of programmed cell death. The encoded protein contains a distinct N-terminal pyrin-like motif, which is possibly involved in protein-protein interactions. This protein interacts strongly with caspase 2 and weakly with caspase 9. Overexpression of this gene was demonstrated to induce apoptosis.
NAPL1 is involved in the regulation of inflammation and response and the regulation of caspase activity.
NAPL1 expression induces apoptosis in cerebellar granule neurons and HeLa cells. The expression of NALP 1 induces activation of caspase 3
NAPL1 expression is regulated by the TF CREB

BAIAP3 BAI1-associated protein 3 [Homo sapiens]

Gene name and aliases: BAIAP3, BAP3; KIAA0734

Gene description: BAI1-associated protein 3. This p53-target gene encodes a brain-specific angiogenesis inhibitor. The protein is a seven-span transmembrane protein and a member of the secretin receptor family. It interacts with the cytoplasmic region of brain-specific angiogenesis inhibitor 1. This protein also contains two C2 domains, which are often found in proteins involved in signal transduction or membrane trafficking. Its expression pattern and similarity to other proteins suggest that it may be involved in synaptic functions.
BAIAP3 was originally identified from a yeast two-hybrid assay searching for proteins interacting with the cytoplasmic domain of brain angiogenesis inhibitor-1 (BAI1).
Ectopic expression of BAIAP3 in tumor cells dramatically enhances growth in low serum and colony formation in soft agar. BAIAP3 therefore encodes a transcriptional target of an oncogenic fusion protein that implicates the regulated exocytotic pathway in cancer cell proliferation.

BP4, BIK BCL2-interacting killer (apoptosis-inducing) [Homo sapiens]

Gene name and aliases: BIK, BP4; NBK; BBCl; BIP1

Gene description: BCL2-interacting killer (apoptosis-inducing). The protein encoded by this gene is known to interact with cellular and viral survival-promoting proteins, such as BCL2 and the Epstein-Barr virus in order to enhance programed cell death. Because its activity is suppressed in the presence of survival-promoting proteins, this protein is suggested as a likely target for antiapoptotic proteins. This protein shares a critical BH3 domain with other death-promoting proteins, BAX and BAK.
Induction of cell death by the BH3-only Bcl-2 homolog Nbk/Bik is mediated by an entirely Bax-dependent mitochondrial pathway.
The pro-apoptotic protein, Bik, exhibits potent antitumor activity that is dependent on its BH3 domain.
Systemic tumor suppression by the proapoptotic gene BIK.
BH-3-only BIK functions at the endoplasmic reticulum to stimulate cytochrome c release from mitochondria.

PEG10 paternally expressed 10)

Name and Alias: Edr; HB-1; MEF3L; KIAA1051-Homologue in the mouse myelin expression factor-3 (MyEF-3) regulates the expression of myelin

Gene description : Imprinted gene , (paternally expressed 10) that seems to play a role in the liver regeneration and carcinogenesis.
Exogenous expression of PEG 10 conferred oncogenic activity. PEG10 protein associated with SIAH1, a mediator of apoptosis, and over expression of PEG10 decreased the cell death mediated by SIAH1
PEG10 is found to be over expressed (RNA level ) in Hepato Cellular Carcinoma and in the regenerating liver of the mouse

BAG2 BCL2-associated athanogene 2 [Homo sapiens]

Gene name and aliases: BAG2, BAG-2

Gene description: BCL2-associated athanogene 2. BAG proteins are highly conserved throughout eukaryotes and regulate Hsc/Hsp70-mediated molecular chaperone activities and apoptosis. The phosphorylation of BAG2 was specifically controlled by a p38 MAPK-dependent manner. Furthermore, BAG2 was directly phosphorylated at serine 20 in vitro by MAPKAP kinase 2, which is known as a primary substrate of p38 MAP kinase and mediates several p38 MAPK-dependent processes.
Proteomic identification of Bcl2-associated athanogene 2 as a novel MAP kinase-activated protein kinase 2 substrate.
BAG3 was also reported as a regulator of stress-induced apoptosis in normal and neoplastic leukocytes.

TNFRSF6 tumor necrosis factor receptor superfamily, member 6 [Homo sapiens]

Gene name and aliases: TNFRSF6, FAS; APT1; CD95; FAS1; APO-1; FASTM

Gene description: tumor necrosis factor receptor superfamily, member. The protein encoded by this gene is a member of the TNF-receptor superfamily. This receptor contains a death domain. It has been shown to play a central role in the physiological regulation of programmed cell death, and has been implicated in the pathogenesis of various malignancies and diseases of the immune system. The interaction of this receptor with its ligand allows the formation of a death-inducing signaling complex that includes Fas-associated death domain protein (FADD), caspase 8, and caspase 10. The autoproteolytic processing of the caspases in the complex triggers a downstream caspase cascade, and leads to apoptosis. This receptor has been also shown to activate NF-kappaB, MAPK3/ERK1, and MAPK8/JNK, and is found to be involved in transducing the proliferating signals in normal diploid fibroblast and T cells. At least eight alternatively spliced transcript variants encoding seven distinct isoforms have been described. The isoforms lacking the transmembrane domain may negatively regulate the apoptosis mediated by the full length isoform.
the selective down-regulation of c-FLIP by small interfering RNA oligoribonucleotides was sufficient to sensitize Hodgkin/Reed-Sternberg cells to CD95 and tumor necrosis factor-related apoptosis-inducing ligand-induced apoptosis
Fas/CD95 pathway is activated by Ad-p53 in human gliomas
CD95 death-inducing signaling complex formation and internalization in type I and type II cells occur in lipid rafts, which are a major site of caspase-8 activation
Fas, DR4, and DR5 are activated in drug-sensitive cells in response to anticancer drugs depending on the cytotoxic effect of each drug
CD95/Fas binds to the expanded binding surface of the FADD death domain

TRIB3 tribbles homolog 3 (Drosophila) [Homo sapiens]

Gene name and aliases: TRIB3, NIPK; SINK; TRB3; SKIP3; C20or f97

Gene description: tribbles homolog 3 (Drosophila). The protein encoded by this gene is a putative protein kinase that is induced by the transcription factor NF-kappaB. The encoded protein is a negative regulator of NF-kappaB and can also sensitize cells to TNF- and TRAIL-induced apoptosis. In addition, this protein can negatively regulate the cell survival serine-threonine kinase AKT1.
results suggest that TRB3, a mammalian homolog of Drosophila tribbles, promotes glucose output from liver under fasting conditions by binding to and interfering with Akt phosphorylation in response to residual insulin signaling [Trb3 tribbles homolog 3]
SINK specifically interacted with the NF-kappaB transactivator p65 and inhibited p65 phosphorylation
This protein, a novel Drosophila tribbles ortholog, is overexpressed in human tumors and is regulated by hypoxia.

BID BH3 interacting domain death agonist

Name and Alias: BID

Gene description: BID is a death agonist that heterodimerizes with either agonist BAX or antagonist BCL2. The encoded protein is a member of the BCL-2 family of cell death regulators. It is a mediator of mitochondrial damage induced by caspase-8 (CASP8);
BID can be cleaved by Caspase 8 or granzyme B, each type of cleavage leads to a preferential interaction (Bax or Bak).
BID is cleaved upon SAHA treatment, independently of caspase activity (T cell leukemia cells). The mechanism was independent of p53
BID has a role as an apoptosis inducer by triggering the oligomerisation but also as a regulator by interacting with anti apoptotic protein such as Bcl-xl

TP53 tumor protein p53 (Li-Fraumeni syndrome) [Homo sapiens]

Gene name and aliases: TP53, p53; TRP53

Gene description: tumor protein p53 (Li-Fraumeni syndrome). Tumor protein p53, a nuclear protein, plays an essential role in the regulation of cell cycle, specifically in the transition from G0 to G1. It is found in very low levels in normal cells, however, in a variety of transformed cell lines, it is expressed in high amounts, and believed to contribute to transformation and malignancy. p53 is a DNA-binding protein containing DNA-binding, oligomerization and transcription activation domains. It is postulated to bind as a tetramer to a p53-binding site and activate expression of downstream genes that inhibit growth and/or invasion, and thus function as a tumor suppressor. Mutants of p53 that frequently occur in a number of different human cancers fail to bind the consensus DNA binding site, and hence cause the loss of tumor suppressor activity. Alterations of the TP53 gene occur not only as somatic mutations in human malignancies, but also as germline mutations in some cancer-prone families with Li-Fraumeni syndrome.
Data suggest that geminin is required for suppressing overreplication in cells with wild-type or mutant p53 and that a G(2)/M checkpoint restricts the proliferation of cells with overreplicated DNA.
Regulation of cyclin E expression plays a role underlying numeral homeostasis of centrosomes in human bladder cancer cells and that deregulation of cyclin E expression, together with inactivation of p53, results in centrosome amplification.
Results demonstrated a novel function of Ser(392) phosphorylation in regulating the oncogenic function of mutant p53.

SSP29, APRIL, ANP32B acidic (leucine-rich) nuclear phosphoprotein 32 family, member B [Homo sapiens]

Gene name and aliases: ANP32B, APRIL; SSP29; PHAPI2

Gene description: acidic (leucine-rich) nuclear phosphoprotein 32 family, member B
production of APRIL and its ability to increase the proliferation of eight human glioblastoma cell lines
B-CLL cells can be rescued from apoptosis through an autocrine process involving BAFF, APRIL, and their receptors.
APRIL up-regulates activation-induced cytidine deaminase and enhances class-switch DNA recombination in Epstein-Barr virus-infected B cells.
regulatory roles of oncoprotein ProT and tumor suppressor PHAP in apoptosis

Cell death regulator Aven apoptosis, caspase activation inhibitor

Gene name and aliases: AVEN, PDCD12

Gene description: apoptosis, caspase activation inhibitor. Aven was discovered in a yeast two-hybrid screen of human B-cell cDNA libraries using a mutant Bcl-xL as bait. Expression of Aven mRNA was detected in a wide variety of adult tissues and cell lines. Also, cells transiently cotransfected with Aven and Bcl-xL revealed that some, but not all, of the Aven and Bcl-xL colocalized. Aven was found to interact with anti-apoptotic Bcl-2 family members in immunoprecipitation studies. Aven may be providing protection early in the apoptosis pathway before or during the signaling of initiator caspase-9 activation. Aven was found to interacts with Apaf-1, a mammalian homolog of CED-4.
Aven and Bcl-xL enhance protection against apoptosis for mammalian cells exposed to various culture conditions.

BCL2L1 BCL2-like 1 [Homo sapiens]

Gene name and aliases: BCL2L1, BCLX; BCL2L; Bcl-X; bcl-xL; bcl-xS; BCL-XL/S

Gene description: BCL2-like 1. The protein encoded by this gene belongs to the BCL-2 protein family. BCL-2 family members form hetero- or homodimers and act as anti- or pro-apoptotic regulators that are involved in a wide variety of cellular activities. The proteins encoded by this gene are located at the outer mitochondrial membrane, and have been shown to regulate outer mitochondrial membrane channel (VDAC) opening. VDAC regulates mitochondrial membrane potential, and thus controls the production of reactive oxygen species and release of cytochrome C by mitochondria, both of which are the potent inducers of cell apoptosis. Two alternatively spliced transcript variants, which encode distinct isoforms, have been reported. The longer isoform acts as an apoptotic inhibitor and the shorter form acts as an apoptotic activator.
Reduced levels of Bcl-xL may play an important role in the increased sensitivity to apoptosis of HIV-specific CD8+T cells.
Bcl-xL in hepatocellular carcinoma specimens. Bcl-xL may be significant prognostic factor for disease progression in human hepatocellular carcinoma.
retinoic acid-induced apoptotic signals were transduced via downregulation of Bcl-xL and the decrease in the mitochondrial membrane function leading to caspase-3 activation
CD28-stimulated T cells actively secrete IL-8, and Bcl-xL up-regulation protects T cells from radiation-induced apoptosis
Bcl-xL and ElB-19K proteins inhibit p53-induced irreversible growth arrest and senescence by preventing reactive oxygen species-dependent p38 activation
Bcl-x(L)-anti-apoptotic signal pathway seems to prevent mitochondrial multiple conductance channel opening, cytochrome c release and caspase-3 like activity following 6-OHDA treatment in the human neuroblastoma cell line SH-SY5Y
bcl-xL directly binds to Apaf-1.

ARK5 AMP-activated protein kinase family member 5 [Homo sapiens]

Gene name and aliases: ARK5

Gene description: AMP-activated protein kinase family member 5, a novel AMPK catalytic subunit family member, plays a key role in tumor malignancy downstream of Akt. ARK5 is the tumor cell survival factor activated by Akt and acts as an ATM kinase under the conditions of nutrient starvation via inhibition of caspase 8 activation.
Strong association of ARK5 with tumor invasion and metastasis.
Regulation of caspase-6 and FLIP by the AMPK family member ARK5.
ARK5 is a tumor invasion-associated factor downstream of Akt signaling.
ARK5 expression in colorectal cancer and its implications for tumor progression.
EGF mediates multiple signals: dependence on the conditions.
ARK5 suppresses the cell death induced by nutrient starvation and death receptors via inhibition of caspase 8 activation, but not by chemotherapeutic agents or UV irradiation.
Identification of a novel protein kinase mediating Akt survival signaling to the ATM protein.

BCL3 B-cell CLL/lymphoma 3 [Homo sapiens]

Gene name and aliases: BCL3, BCL4; D19S37

Gene description: B-cell CLL/lymphoma 3. This gene is a proto-oncogene candidate. It is identified by its translocation into the immunoglobulin alpha-locus in some cases of B-cell leukemia. The protein encoded by this gene contains seven ankyrin repeats, which are most closely related to those found in I kappa B proteins. This protein functions as a transcriptional co-activator that activates through its association with NF-kappa B homodimers. The expression of this gene can be induced by NF-kappa B, which forms a part of the autoregulatory loop that controls the nuclear residence of p50 NF-kappa B.
BCL3 is downregulated by p53 to repress cyclin D1 and has a role in regulation of cell cycle progression
High-level expression of BCL3 differentiates t(2;5)(p23;q35)-positive anaplastic large cell lymphoma from Hodgkin disease.

BHLHB2 basic helix-loop-helix domain containing, class B, 2 [Homo sapiens]

Gene name and aliases: BHLHB2, DEC1; STRA13; Stra14

Gene description: basic helix-loop-helix domain containing, class B2. DEC1 encodes a basic helix-loop-helix protein expressed in various tissues. Expression in the chondrocytes is responsive to the addition of Bt2cAMP. Differentiated embryo chondrocyte expressed gene 1 is believed to be involved in the control of cell differentiation.
DEC1-mediated anti-apoptosis is achieved by blocking apoptotic pathways initiated via the mitochondria. The results functionally distinguish DEC1 from other bHLH proteins and directly link this factor to oncogenesis.
DEC1 -mediated repression on the expression of DEC2 provides an important mechanism that these transcription factors regulate the cellular function of members within the same class
DEC1 is the first transcription factor that can promote both chondrogenic differentiation and terminal differentiation
DEC1 and DEC2 may play a crucial role in the adaptation to hypoxia
Dec1 and Dec2 are regulators of the mammalian molecular clock, and form a fifth clock-gene family.

What is claimed is:

1. A method for screening for HDAC activity in cells in vitro comprising
a). detecting expression levels of any one or more genes selected from the group consisting of those disclosed in Table 1 in said cells and in control cells; and,
b). comparing expression levels from said cells with expression levels from control cells
wherein a difference in expression levels in said cells relative to the expression levels of corresponding genes or proteins in the control cells indicate that said cells contain HDAC activity.

2. The method of claim 1 wherein said expression levels of said gene is the level of mRNA.

3. The method of claim 1 wherein said expression level is the level of a protein encoded by said genes.

4. A method for screening for HDAC inhibition in a subject in vivo comprising
a). detecting expression levels of any one or more genes selected from the group disclosed in Table 1 in said subject in vivo and in said control subject known not to possess conditions related to abnormal HDAC activity; and,
b). comparing said expression levels in said subject in vivo with expression levels in said control subject wherein a difference in expression levels between said subject and said control subject indicates that HDAC activity is inhibited in said subject in vivo.

5. The method of claim 4 wherein said expression levels of said gene is the level of mRNA.

6. The method of claim 4 wherein said expression level is the level of a protein encoded by said gene.

7. A method for screening a compound for HDAC inhibitory activity in vitro, comprising
a). administering a compound to cells in vitro to obtain treated cells;
b). assaying for expression levels of any one or more genes selected from the group consisting of those disclosed in Table 1 in said treated cells and in control cells to which no compound has been administered; and,
c) comparing said expression levels between said treated cells and said control cells wherein a difference in expression levels between said treated cells and control levels indicates whether said compound possesses HDAC inhibitory activity.

8. The method of claim 7 wherein said expression levels is the level of RNA.

9. The method of claim 7 wherein said expression level is the level of protein encoded by said genes.

10. A method for screening a compound for HDAC inhibitory activity in a subject in vivo, comprising
a). assaying expression levels of any one or more genes selected from the group disclosed in Table 1 in said subject;
b). administering a compound to said subject;
c). reassaying expression levels in said subject after administration of the compound; and,
d) comparing expression levels in said subject before and after administration of the compound wherein a difference in expression levels in said subject before and after administration of the compound indicates said compound possesses HDAC inhibitory activity.

11. The method of claim 10 wherein said expression levels is the level of mRNA.

12. The method of claim 4 wherein said expression levels is the level of protein encoded by said genes.

13. A method for monitoring the therapeutic efficacy of a known HDAC inhibitor in a subject comprising
a). detecting expression levels of any one or more genes selected from the group disclosed in Table 1 in said subject before and after treatment with said HDAC inhibitor; and,
b). comparing said expression levels in said subject wherein a difference in expression levels in said subject after treatment compared to expression levels before treatment indicate that the HDAC inhibitor is therapeutically effective.

14. The method of claim 13 wherein genes selected from the group consisting of those disclosed in Table 2.

15. The method of claim 14 wherein said the expression levels are levels of proteins encoded by said genes.

16. A method to determine the sensitivity of a cell to a known HDAC inhibitor comprising
a). administering said HDAC inhibitor to said cells in vitro; and,
b). screening for expression levels of any one or more genes selected from the group disclosed in Table 1 in said cells and in control cells to which no HDAC inhibitor has been administered; and,
c). comparing expression levels between said cells and said control cells wherein a difference in expression levels of any one or more genes disclosed in Table 1 indicates the sensitivity of said cells to said HDAC inhibitor.

17. The method of claim 16 wherein an absence of expression of one or more genes or proteins encoded by genes disclosed in Table 1 indicates resistance of said cells to said HDAC inhibitor.

18. The method of claim 16 wherein said expression level is the level of mRNA in said cells.

19. A method to determine resistance of a subject to a known HDAC inhibitor comprising
a). detecting expression levels of any one or more genes or proteins encoded by genes selected from the group disclosed in Table 1 in said subject before and after treatment with said HDAC inhibitor; wherein lack of expression of any one or more genes or proteins encoded by genes selected from those disclosed in Table 1 indicates resistance of said subject to said HDAC inhibitor.

20. The method of claim 19 wherein said expression levels is the level of mRNA.

21. The method of claim 19 wherein said expression level is the level of protein.

22. A method for treating a condition in a subject, wherein the condition is one for which administration of HDAC inhibitors is indicated, comprising the steps of:
(a) administering a compound to the subject;
(b) obtaining the gene expression profile of the subject, wherein the gene expression profile comprises the gene expression pattern of one or more genes, where the expression patterns of the one or more genes are a consequence of administration of the compound; and
(c) comparing the gene expression profile of the subject to whom the compound was administered to a biomarker gene expression profile indicative of efficacy of treatment by an HDAC inhibitor, wherein a similarity in the gene expression profile of the subject to whom the compound was administered to the biomarker gene expression profile is indicative of efficacy of treatment with the compound.

23. The method of claim 22, wherein the subject is a mammal.

24. The method of claim 22 wherein the HDAC inhibitor is LAQ824.

25. The method of claim 22, wherein the biomarker gene expression profile is the baseline gene expression profile of the subject before administration of the compound.
